(19) United States
(12) Patent Application Publication
(10) Pub. No.: US 2022/0255356 A1
(43) Pub. Date: Aug. 11, 2022

(54) BIOCOMPATIBLE SENSOR DEVICE AND METHOD FOR DETECTING ENVIRONMENTAL STIMULI THEREWITH

(71) Applicant: THE REGENTS OF THE UNIVERSITY OF CALIFORNIA, Oakland, CA (US)

(72) Inventors: Peter Tseng, Oakland, CA (US); Manik Dautta, Oakland, CA (US); Muhannad Alshetaiwi, Oakland, CA (US)

(73) Assignee: THE REGENTS OF THE UNIVERSITY OF CALIFORNIA, Oakland, CA (US)

(21) Appl. No.: 17/628,868

(22) PCT Filed: Jul. 22, 2020
(86) PCT No.: PCT/US2020/043142

§ 371 Date: Jan. 20, 2022

Related U.S. Application Data
(60) Provisional application No. 62/877,460, filed on Jul. 23, 2019.

(51) Int. Cl. H02J 50/12; H04B 5/00

(52) U.S. Cl. CPC H02J 50/12; H04B 5/0075; H04B 5/0037

(57) ABSTRACT

A sensor device includes an inner hydrogel layer, a first planar metallic structure adjacent to a first surface of the inner hydrogel layer, and a second planar metallic structure adjacent to a second surface of the inner hydrogel layer opposite to the first surface. A sensor device further includes an encasing layer at least partially enclosing at least one of the inner hydrogel layer, the first planar metallic structure, and the second planar metallic structure. A method for use of the sensor device includes receiving at least one environmental stimulus, modifying a capacitance of a hydrogel in response to the received environmental stimulus, and generating an electrical stimulus response based on the modified capacitance. The method further includes modifying the capacitance of the hydrogel by modifying a resonant frequency of the hydrogel.

### CROSS-REFERENCE TO RELATED PATENT APPLICATIONS

This application claims priority is a 371 National Stage Entry of International Application No. PCT/US2020/043142, filed Jul. 22, 2020, which claims the domestic benefit under Title 35 of the United States Code § 119(e) of U.S. Provisional Patent Application Ser. No. 62/877,460, entitled “Fabrication and Read-Out from Selective to Non-Selective, Interlayer-Radio-Frequency Sensors,” filed Jul. 23, 2019, the contents of such application being hereby incorporated by reference in its entirety and for all purposes as if completely and fully set forth herein.

### TECHNICAL FIELD

The present embodiments relate generally to electronic sensors, and more particularly to a biocompatible sensor device and a method for detecting environmental stimuli therewith.

### BACKGROUND

Biosensing technologies are of broad interest across engineering communities due to their ability to monitor living systems. Such sensors are critical to enhancing understanding of complex biological environments, including the human body, and enable tracking of constantly fluctuating states within. This need is ever more present as new, enabling technologies in machine learning, computation, and the Internet-of-Things may process an ever-increasing amount of information about the environment. Analytical biosensors, however, often fail to live up to the rigorous demands of modern systems that require flexible, small, and long-term wireless tags for continuous monitoring of demanding, aqueous environments. Passive wireless sensors targeting different scales and supporting multi-functionality in sensing environmental stimuli could transform healthcare. Thus, there is a tremendous need to track chemical moieties that make up the many fluids of our environment, as the monitoring of these systems can indicate the presence or balance of toxins, drugs, proteins, ions, carbohydrates, and more.

Non-invasive medical devices can be powerful tools to provide data on and track human performance, biomarkers, and wellness. Within this class of devices, conformal interfaces in the form of flexible devices and tattoos 1-15 can provide lower form factor than traditional medical devices, reduced user burden, and their ability to monitor physiological parameters on complex surfaces/environments, including glucose, heartbeat, and acceleration. Significant potential exists in advancing mechanical performance, micro-electronic processors, and encompassing microfluidic microsystems of such devices. Yet, conventional analytical biosensors at the core of such systems, including medical devices, have lagged behind. Conventional electrochemical sensors have fundamental limitations such as heavy signal drift, dependence on degradative reference electrodes, and bulky wireless formats that prevent many applications, including those requiring long-term/continuous read-out, wireless function, tracking of complex biofluidic environments, and more. Thus, there exists a technological need for new versatile, sensor architectures with various properties. Chief among these properties are stretchability, long-term stability, small size, diverse analytical sensing, and wireless formats with minimal connected electronics. Such capabilities would facilitate devices that could integrate seamlessly with complex environments, while providing important information read-out over long time-scales.

### SUMMARY

Sensor devices in accordance with present embodiments are configurably responsive to a wide range of environmental stimuli. Exemplary devices, systems, and arrays blur the line between material and device as multi-functional layers merge to create continuous materials capable of multi-signal, passive environmental read-out without integrated batteries and/or micro-electronics. An exemplary sensor device thus includes an inner hydrogel layer, a first planar metallic structure adjacent to a first surface of the inner hydrogel layer, and a second planar metallic structure adjacent to a second surface of the inner hydrogel layer opposite to the first surface. A sensor device further includes an encasing layer at least partially enclosing at least one of the inner hydrogel layer, the first planar metallic structure, and the second planar metallic structure. A method for use of the sensor device includes receiving at least one environmental stimulus, modifying a capacitance of a hydrogel in response to the received environmental stimulus, and generating an electrical stimulus response based on the modified capacitance. The method further includes modifying the capacitance of the hydrogel by modifying a resonant frequency of the hydrogel. The method further includes modifying the signal magnitude response of the hydrogel by shifting the signal magnitude response relative to the resonant frequency of the hydrogel. A method for manufacturing the sensor device includes forming a plurality of planar metallic structures, forming an inner hydrogel, and affixing the inner hydrogel between the planar metallic structures.

### BRIEF DESCRIPTION OF THE DRAWINGS

These and other aspects and features of the present embodiments will become apparent to those ordinarily skilled in the art upon review of the following description of specific embodiments in conjunction with the accompanying figures, wherein:

FIG. 1A illustrates an exemplary split-ring sensor device in accordance with present embodiments.

FIG. 1B illustrates an exploded view of the exemplary sensor device of FIG. 1A.

FIG. 1C illustrates a cross-sectional view of an exemplary encapsulated sensor device in accordance with present embodiments.

FIG. 1D illustrates the exemplary sensor device of FIG. 1A receiving exemplary stimuli and generating an exemplary response, in accordance with present embodiments.

FIG. 1E illustrates an exemplary sensor device including an electronic device, in accordance with present embodiments.

FIG. 1F illustrates the exemplary sensor device of FIG. 1E receiving exemplary stimuli and generating an exemplary response, in accordance with present embodiments.

FIG. 2A illustrates an exemplary planar-coil sensor device including an electronic device, in accordance with present embodiments.

FIG. 2B illustrates an exploded view of the exemplary sensor device of FIG. 2A.

FIG. 2C illustrates a cross-sectional view of an exemplary encapsulated sensor device in accordance with present embodiments.

FIG. 2D illustrates the exemplary sensor device of FIG. 2A receiving exemplary stimuli and generating an exemplary response, in accordance with present embodiments.

FIG. 3 illustrates an exemplary sensor circuit in accordance with present embodiments.

FIG. 4A illustrates an exemplary surface-mountable, wearable, or like sensor system including the exemplary sensor device of FIG. 1A, in accordance with present embodiments.

FIG. 4B illustrates an exemplary embeddable, implantable, or like sensor system including the exemplary sensor device of FIG. 1A, in accordance with present embodiments.

FIG. 5A illustrates an exemplary resonant frequency response to pressure stimulus of an exemplary sensor device in accordance with present embodiments.

FIG. 5B illustrates an exemplary resonant frequency response to temperature stimulus of an exemplary sensor device in accordance with present embodiments.

FIG. 5C illustrates an exemplary resonant frequency response to ionic hydrogen, pH, or like stimulus of an exemplary sensor device in accordance with present embodiments.

FIG. 5D illustrates an exemplary signal magnitude response to salt (NaCl) stimulus of an exemplary sensor device in accordance with present embodiments.

FIG. 6 illustrates an exemplary change to response magnitude relative to resonant frequency, in response to exemplary stimuli of an exemplary sensor device in accordance with present embodiments.

FIG. 7 illustrates an exemplary change to response magnitude relative to resonant frequency, in response to further exemplary stimuli of an exemplary sensor device in accordance with present embodiments.

FIG. 8 illustrates an exemplary change to response magnitude relative to resonant frequency, in response to multiple exemplary stimuli of an exemplary sensor system in accordance with present embodiments.

FIG. 9 illustrates an exemplary method of manufacturing an exemplary sensor device in accordance with present embodiments.

FIG. 10 illustrates an exemplary method of manufacturing an exemplary sensor device further to the exemplary method of FIG. 9.

FIG. 11 illustrates an exemplary method of manufacturing an exemplary sensor device further to the exemplary method of FIG. 10.

FIG. 12 illustrates an exemplary method of operation of an exemplary sensor device in accordance with present embodiments.

### DETAILED DESCRIPTION

The present embodiments will now be described in detail with reference to the drawings, which are provided as illustrative examples of the embodiments so as to enable those skilled in the art to practice the embodiments and alternatives apparent to those skilled in the art. Notably, the figures and examples below are not meant to limit the scope of the present embodiments to a single embodiment, but other embodiments are possible by way of interchange of some or all of the described or illustrated elements. Moreover, where certain elements of the present embodiments can be partially or fully implemented using known components, only those portions of such known components that are necessary for an understanding of the present embodiments will be described, and detailed descriptions of other portions of such known components will be omitted so as not to obscure the present embodiments. Embodiments described as being implemented in software should not be limited thereto, but can include embodiments implemented in hardware, or combinations of software and hardware, and vice-versa, as will be apparent to those skilled in the art, unless otherwise specified herein. In the present specification, an embodiment showing a singular component should not be considered limiting; rather, the present disclosure is intended to encompass other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present embodiments encompass present and future known equivalents to the known components referred to herein by way of illustration.

Exemplary sensor devices and systems in accordance with present embodiments include, in some embodiments, stacked and resonant-coupled split ring resonators or multi-turn antennas including one or more multi-functional, “smart” interlayers that absorb, swell, or deform in response to environmental stimuli. In some embodiments, a capacitance of an exemplary structure becomes dominated by the behavior of the interlayer material. In some embodiments, an exemplary structure includes one or more membranes, separators, deformable materials, polymers, or the like. In some embodiments, the polymers are responsive to pressure, temperature, pH, chemicals, biochemicals, metals, or the like. In some embodiments, a long-lasting coupled split ring sensor device or system is integrated with a wide range of bio-responsive materials. In some embodiments, an exemplary sensor device or system is attached, implanted, affixed, or the like to new areas, including to a live biological organism including but not limited to the human body. In some embodiments, an exemplary sensor device or system attached, implanted, affixed, or the like to a biological organism to wirelessly monitor complex environments over long durations. Thus, in some embodiments, an exemplary sensor device or system operates passively and wirelessly without any connected microelectronics or batteries. Embodiments in accordance with present embodiments therefore enable, among other advantages, long-lifetime, microelectronics-free operation, and inherent biocompatibility for improved and novel applications in human health tracking and management. In some embodiments, hydrogels interact with living cells/tissues, and membranes and films extract desired biomolecules from complex biofluids to enable a plurality of read-outs, analyses, or the like associated with biological systems. Thus, exemplary sensors in accordance with present embodiments embed directly into long-lasting, soft constructs that possess fully wireless, multi-parametric read-out, and provide properties of both stretchable materials and functional devices.

Exemplary sensor devices and systems, in some embodiments, are completely passive and fully biocompatible. They operate remotely and wirelessly without any integrated power supply. In some embodiments, an exemplary sensor can read out signal passively. In some embodiments, an exemplary sensor can be implanted in a variety of locations in the body and read out at least one analytical state. In some embodiments, an exemplary sensor can report the analytical state via an integrated circuit or remotely via wireless communication. In addition, interlayers and materials associated therewith in accordance with present embodiments have long useful lifetimes and do not materially degrade over desirable useful lifetimes. In some embodiments, an exemplary structure is combined with one or more dielectric (RF) sensors with inherent long-lasting useful lifetime and biocompatibility properties. An exemplary sensor device or system, in some embodiments, senses the presence of analytes via permittivity shifts in the environment (“label-free” biosensors), and can be built into radio-frequency formats via structuring in the form of an antenna. In some embodiments, permittivity shifts caused by environmental changes modulate a capacitance of the sensor, which changes a spectral response of the sensor via a shift in magnitude of response or resonant frequency.

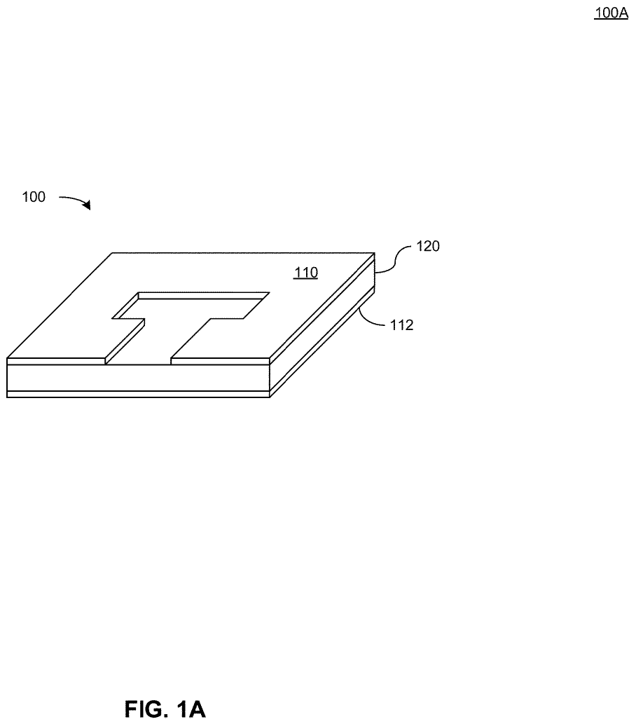

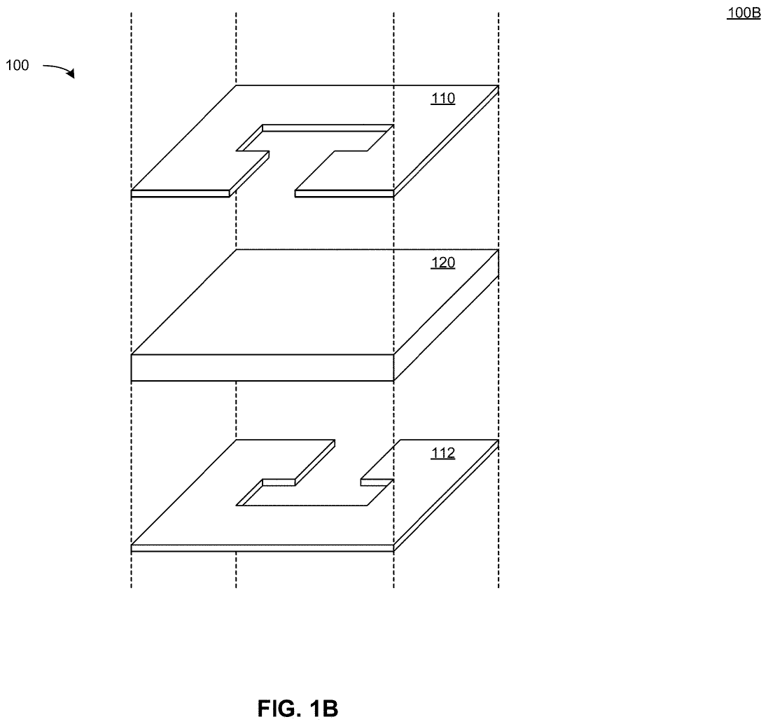

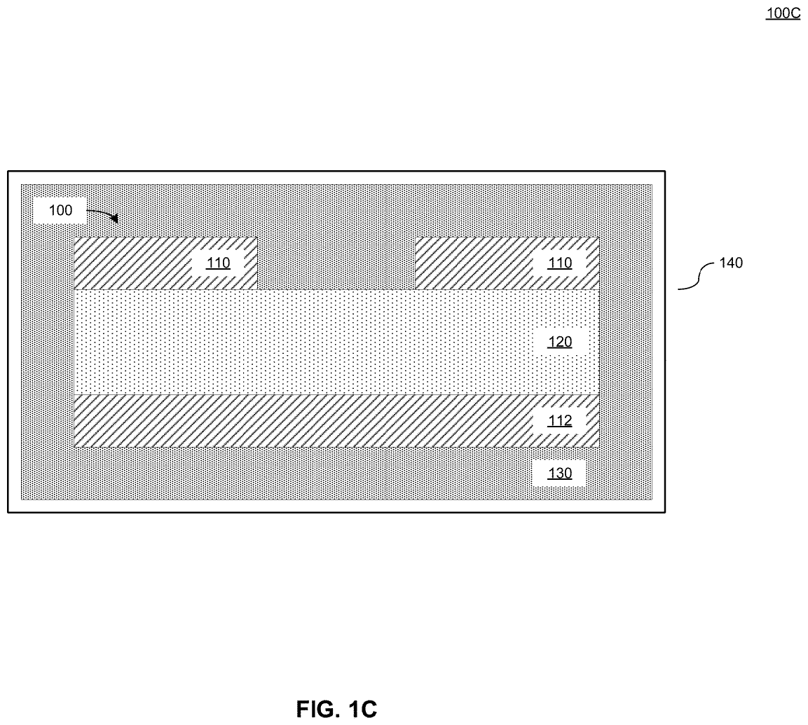

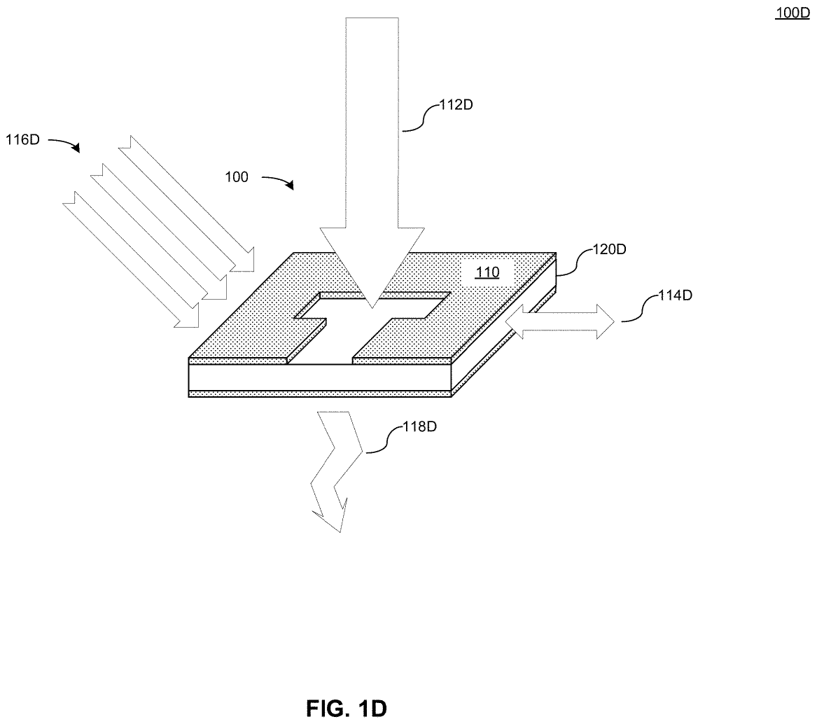

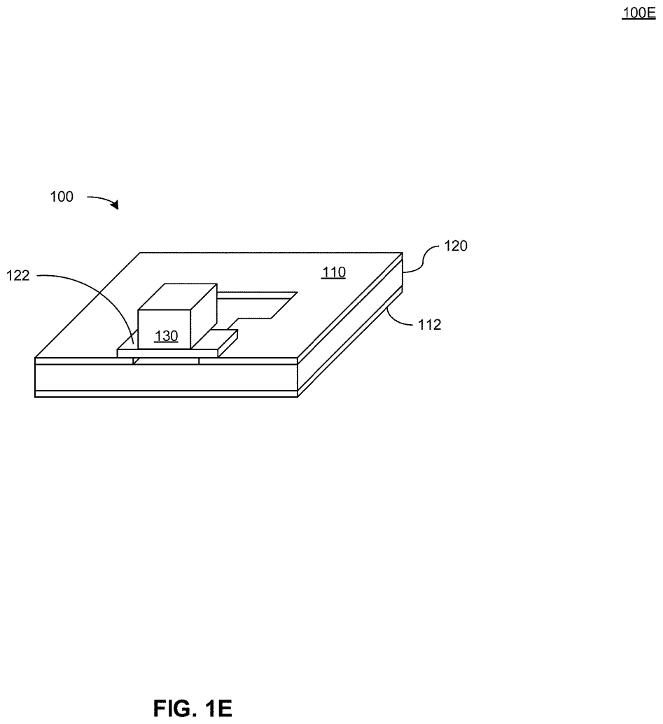

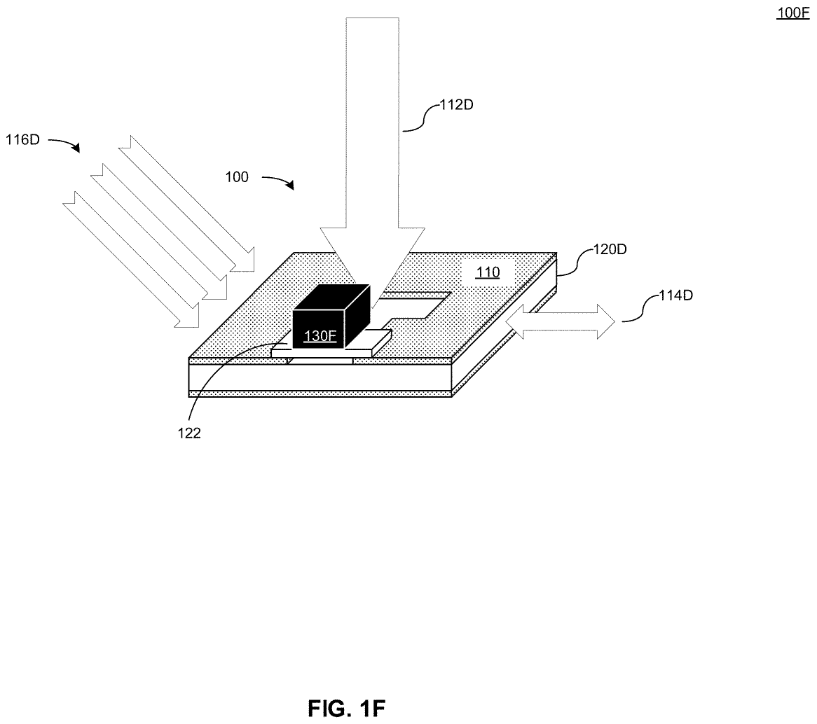

FIG. 1A illustrates an exemplary split-ring sensor device 100 in accordance with present embodiments. As illustrated in view 100A, an exemplary split-ring sensor device includes an upper split-ring metallic structure 110, a lower spilt-ring metallic structure 112, and an interlayer 120 disposed between the upper and lower split-ring metallic structures 110 and 112. In some embodiments, the device 100 is capable of reacting to a desired analyte, converting the reaction to an electrical signal, and converting the electrical signal into information indicating presence or concentration of the desired analyte. In some embodiments, the device 100 monitors analytes using one or more mechanical, optical, electrochemical and electromagnetic processes or devices. In some embodiments, mechanical devices include resonators and piezoelectric devices or the like. In some embodiments, optical processes include spectroscopic, plasmonic, or like processes. In some embodiments, electromagnetic devices include waveguides, dielectric devices, bio-field effect transistors, or the like. In some embodiments, the exemplary device 100 operates in NFC frequency bands. In some embodiments, the sensor device 100 comprises a radio-frequency identification (“RFID”) device, or a passive RFID device capable of biosensing.

The upper and lower split-ring metallic structures 110 and 112 comprise electrical resonators. In some embodiments, the upper and lower split-ring metallic structures 110 and 112 are configured in passive or active formats and probed remotely. In some embodiments, remote probing comprises inductive coupling with a device external to the device 100, via one or more of the structures 110 and 112. In some embodiments, an environmental signal modulates a spectral response of the structures 110 and 112 as an antenna. In some embodiments, the spectral response is readable remotely via an external device. In some embodiments, the sensor device 100 operates at lower frequencies by adding turns to at least one of the upper and lower split-ring metallic structures 110 and 112.

The interlayer 120 is disposed between the upper and lower split-ring metallic structures 110 and 112, and has a capacitance variable in response to environmental stimulus or stimuli. In some embodiments, the interlayer 120 is bio-functional, bio-responsive, bio-compatible, or the like. In some embodiments, an environmental stimulus to the interlayer 120 causes the environmental signal through a shifting capacitance. In some embodiments, the interlayer 120 is partially selective with respect to a plurality of compatible environmental stimuli. Thus, in some embodiments, the interlayer 120 enables RF biosensors with programmable sensitivity and selectivity to diverse environmental stimuli.

In some embodiments, capacitance of the interlayer 120 is at least partially dependent on at least one of the interlayer thickness and permittivity between the upper and lower split-ring metallic structures 110 and 112. As one example, at small thicknesses parasitic capacitance is minimal, minimizing interference with a primary sensing signal. Resonant frequency, or read-out, of the exemplary sensor device 100 is at least partially based on at least one of composition and behavior of the interlayer 120. In some embodiments, the interlayer 120 swells or absorbs chemical or biochemical analyte in response to environmental stimulus. In some embodiments, swelling or contraction of the interlayer 120 due to interaction with chemical, biochemical, temperature, pressure or like stimulus modifies resonant responses of the sensor device 100. Interlayer-dependent capacitance in accordance with present embodiments is easily scalable to both dominate sensor signal and reduce signal operating frequency by reducing interlayer thickness.

In some embodiments, the interlayer 120 comprises a hydrogel. In accordance with present embodiments, the interlayer 120 can comprise various materials with properties including, but not limited to, being deformable, environmentally-responsive, absorptive, degradation-resistant, or the like. In some embodiments, the interlayer 120 is capable of analytical biosensing in aqueous environments. In some embodiments, the interlayer 120 itself comprises water or includes water content. Water has high permittivity (εr=80) and forms an effective ultra-k dielectric. In some embodiments, when combined with scaling of thickness of the interlayer 120, the sensor 120 is compatible with a wide range of operating frequencies and scales and is at least electrically compatible with tissue implantation. In some embodiments, sensor devices in accordance with present embodiments scale down to sub-10 μm while possessing frequency responses in the gigahertz (GHz) range.

FIG. 1B illustrates an exploded view of the exemplary sensor device of FIG. 1A. As illustrated in view 100B, the interlayer 120 is disposed between the metallic structures 110 and 112. In some embodiments, the interlayer 120 is sandwiched between the metallic structures 110 and 112.

FIG. 1C illustrates a cross-sectional view of an exemplary encapsulated sensor device in accordance with present embodiments. As illustrated in view 100C, the exemplary sensor device includes encapsulating outer hydrogel 130 and encapsulating membrane 140. In some embodiments, the encapsulating outer hydrogel 130 at least partially surrounds the exemplary sensor device 100. In some embodiments, the exemplary sensor device 100 is embedded within at least one of an additional hydrogel and a silicone layer. It is to be understood that materials with properties compatible with those of the outer hydrogel 130 may, in accordance with present embodiments, be combined, integrated, or the like with the outer hydrogel 130, or substituted therefor.

In some embodiments, the encapsulating membrane 140 at least partially encloses one or both of the sensor device 100 and the outer hydrogel 130. Certain hydrogels in accordance with present embodiments must remain hydrated in order to stay functional. Maintaining a sufficiently hydrated environment for this subset of exemplary hydrogels mitigates changes in structure or performance. In some embodiments, the encapsulating membrane 140 provides an isolated, sealed, or like ambient environment surrounding an exemplary sensor device, to prevent evaporation of water content within the outer hydrogel 130. In some embodiments, the encapsulating membrane 140 includes ecoflex or silicone, which do not allow the penetration of water. In some embodiments, sensors encapsulated with the encapsulating membrane 140 can stay responsive even within complex drying or biofluidic environments. In some embodiments, silicone-encapsulated devices retain a consistent resonant frequency over extended periods of time. In some embodiments, the encapsulating membrane 140 is advantageously used for long-term encapsulation to extend the useful lifetime of sensors responsive to various environmental stimuli, in accordance with present embodiments. In some embodiments, the encapsulating membrane 140 directly attaches covalently to the outer hydrogel 130. Alternatively, in some embodiments, the encapsulating membrane 140 encapsulates one or more exemplary sensor devices with a layer of water as buffer in addition or in place of the outer hydrogel 130. The encapsulating membrane can act synergistically with interlayer-RF sensor devices responsive to a wide range of environmental stimuli. In some embodiments, encapsulation removes potential failures including preventing detachment of metallic structures 110 and 112 from the interlayer 120, buffering the metallic structures 110 and 112 from damage, and preventing transfer of fluids in or out of the sensor device 100 to prevent device dehydration and improve sensor specificity.

FIG. 1D illustrates the exemplary sensor device of FIG. 1A receiving exemplary stimuli and generating an exemplary response, in accordance with present embodiments. As illustrated in view 100D, in some embodiments, the exemplary sensor device 100 receives one or more of pressure stimulus 112D, chemical or biochemical stimulus 114D, and temperature stimulus 116D. In some embodiments, the exemplary sensor device 100 generates an electrical response 118D in response to one or more of the stimuli 112D, 114D and 116D. The electrical response 118D includes but is not limited to one or more of a passive inductive response, an induced electrical current response, an electrical response of the electronic device generated through an electrical coupling of the sensor device 100 thereto, or the like.

FIG. 1E illustrates an exemplary sensor device including an electronic device, in accordance with present embodiments. As illustrated in view 100E, in some embodiments, an exemplary electronic sensor device 100 includes an electronic device 130 affixed to the upper split-ring metallic structure 110. In some embodiments, the electronic device 130 is affixed to the upper split-ring metallic structure 110 via at least one electrical contact 122. The electronic device 130 provides additional communication, storage, processing or like capability to the exemplary sensor device 100. In some embodiments, the electronic device 130 includes an LED affixed to the sensor device 100. In some embodiments, the sensor device 100 monitors context-dependent power transfer into biosensing antennas at a resonant frequency at or near NFC frequency (13.56 MHz). In some embodiments, the intensity of light generated by the LED is based on resonant response of the exemplary sensor device in response to one or more environmental stimuli. In some embodiments, the electronic contact 122 comprises a metallic structure or structure including conductive properties. In some embodiments, the electronic contact is affixed to, deposited on, or otherwise connected to the sensor device 100 as is known or may become known.

In some embodiments, the electronic device 130 is responsive to environmental stimuli. As one example, the electronic device 130 is responsive to environmental glucose concentration through the intensity of light, by for example, using context-dependent wireless power transfer via inductive coupling between an active external electrical energy source and the sensor device 100 including the electronic device 130. Wireless power is initially coupled to the sensor device 100 with a single LED, as the electronic device 130, attached across the ends the upper split-ring metallic structure 110. The frequency of this received inductive power is by way of example above the resonant frequency of the sensor. Thus, as the sensor swells and shifts to a higher frequency due to the presence of glucose, the transmitted power more efficiently transfers into the circuit. This type of scheme can be read-out through transparent or partially transparent layers. In some embodiments, transparent or partially transparent layers include biological skin of a human or animal. In some embodiments, LED illuminance can be readily detected through intervening thin biological structures.

FIG. 1F illustrates the exemplary sensor device of FIG. 1E receiving exemplary stimuli and generating an exemplary response, in accordance with present embodiments. As illustrated in view 100F, in some embodiments, the electronic device 130 changes into a state represented by electronic device 130F, in response to one or more of the pressure stimulus 112D, the chemical or biochemical stimulus 114D, and the temperature stimulus 116D. In some embodiments, the exemplary sensor device 100 resonate outside of NFC range in response to the pressure stimulus 112D. As one example, the application of a light finger press to upper split-ring metallic structure 110, resonant frequency of the sensor device 100 into NFC range to cause power coupling with the an NFC reader and subsequent LED activation. In some embodiments, the presence of saline reduces the magnitude of the sensor by increasing the loss of the system, and hence reduces input and emitted power of LEDs when applied to an exemplary sensor. In some embodiments, LED luminance correlates strongly with resonant behavior of a sensor device in accordance with present embodiments. As one example, as the sensor responds to a step concentration of glucose (500 mg/dL), the illuminance of the exemplary sensor device 100 increases linearly before nearing saturation at approximately 1 hour. In some embodiments, this response time is associated with exemplary sensor devices operating at room temperature.

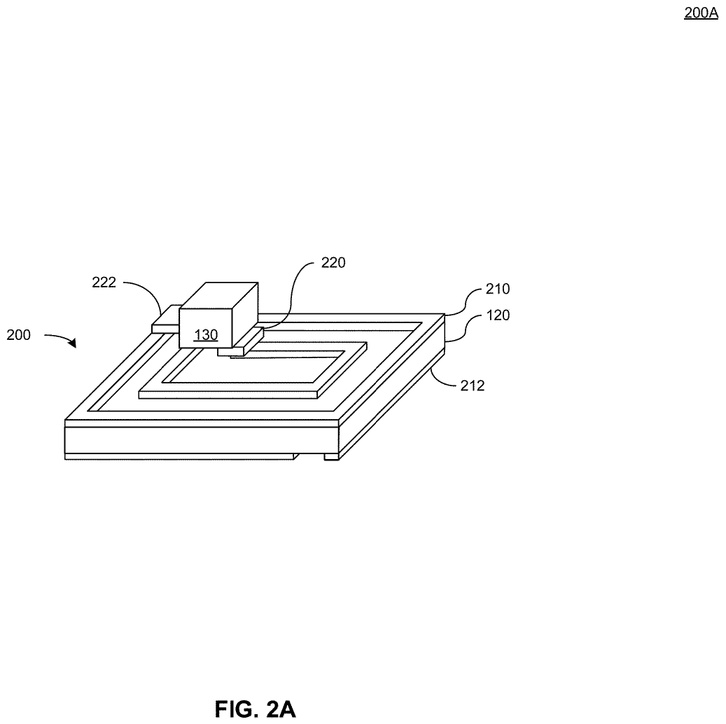

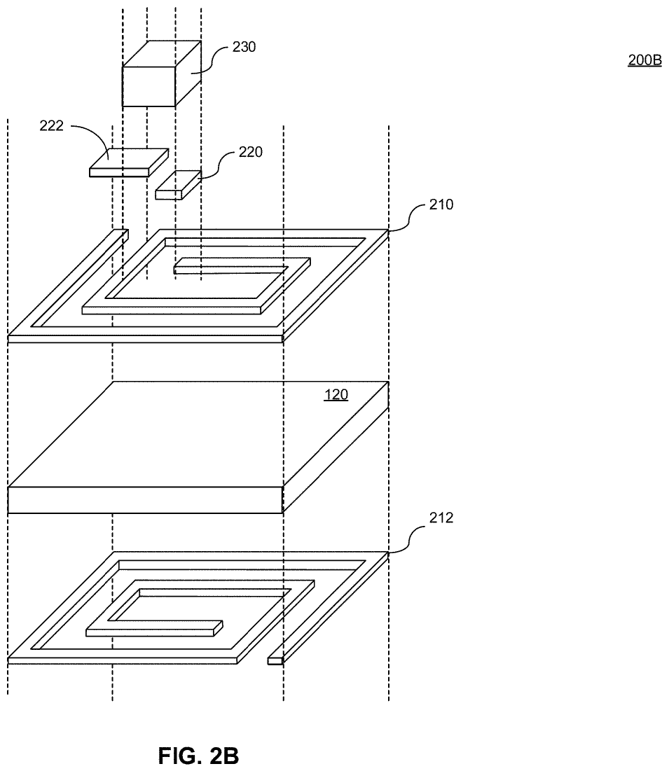

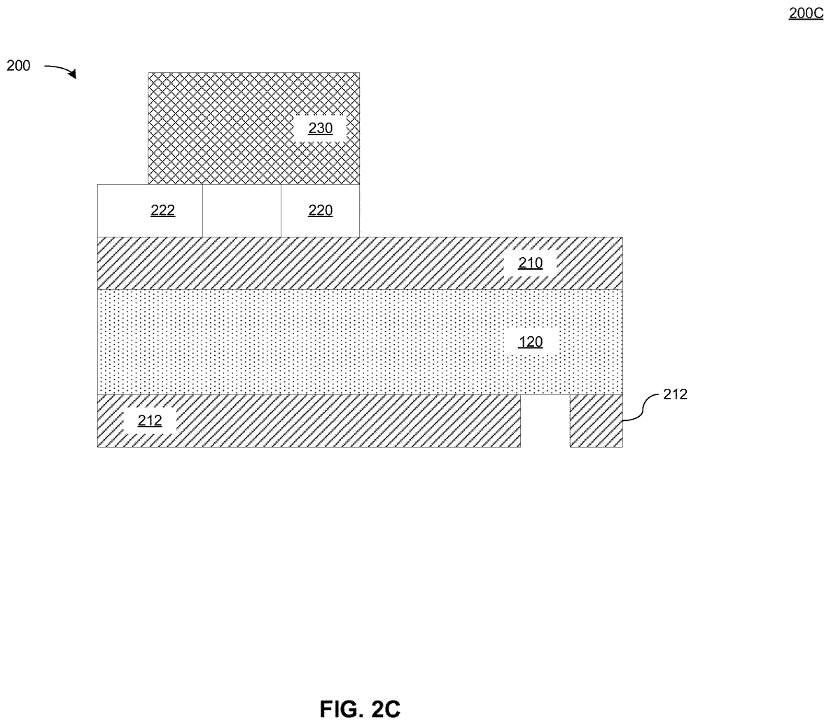

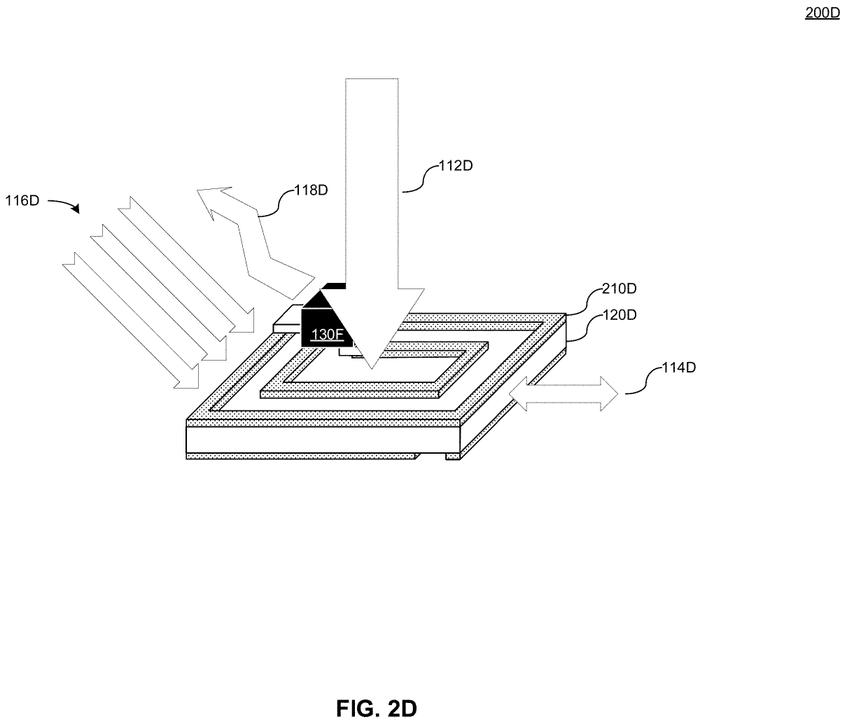

FIG. 2A illustrates an exemplary planar-coil sensor device including an electronic device, in accordance with present embodiments. As illustrated in view 200A, an exemplary planar-coil sensor device 200 includes an upper planar-coil metallic structure 210, a lower planar-coil metallic structure 212, and an interlayer 120 disposed between the upper and lower planar-coil metallic structures 210 and 212. In some embodiments, the exemplary electronic sensor device 200 includes the electronic device 130 affixed to the upper planar-coil metallic structure 210. In some embodiments, the electronic device 130 is affixed to the upper planar-coil metallic structure 210 via at least one electrical contact. In some embodiments, the at least one electrical contact includes an inner coil terminal contact 220 and an outer coil terminal contact 222. The upper and lower planar-coil metallic structures 210 and 212 possess properties similar to those of the split-ring metallic structures 110 and 112, and are similarly responsive to environmental stimuli. Each of the planar-coil metallic structures 210 and 212 includes an inner coil terminal disposed proximate to a center of a planar face of the structure and an outer coil terminal disposed proximate to an edge of the planar face of the structure. The inner coil terminal contact 220 and the outer coil terminal contact 222 are disposed on the upper planar-coil metallic structure 210. In some embodiments, the inner coil terminal contact 220 and the outer coil terminal contact 222 are disposed in electrical contact respectively with the inner coil terminal and the outer coil terminal of the upper planar-coil metallic structure 210.

FIG. 2B illustrates an exploded view of the exemplary sensor device of FIG. 2A. As illustrated in view 100B. As illustrated in view 200B, the interlayer 120 is disposed between the metallic structures 210 and 212. In some embodiments, the interlayer 120 is sandwiched between the metallic structures 210 and 212. It is to be understood that the exemplary planar-coil sensor device 200 may alternatively be operably constructed without one or more of the electronic device 130, the inner coil terminal contact 220, and the outer coil terminal contact 222. In this alternate exemplary embodiment, the exemplary sensor device 200 possess properties similar to those of the exemplary sensor device 100.

FIG. 2C illustrates a cross-sectional view of an exemplary encapsulated sensor device in accordance with present embodiments. As illustrated in view 200C, the exemplary sensor device 200 includes interlayer 120 disposed between the upper planar-coil metallic structures 210 and 212. In some embodiments, the exemplary sensor device 200 includes the electronic device 130, affix, coupled, or the like, to the upper planar-coil metallic structure 210 via the inner and outer coil terminal contacts 220 and 222.

FIG. 2D illustrates the exemplary sensor device of FIG. 2A receiving exemplary stimuli and generating an exemplary response, in accordance with present embodiments. As illustrated in view 200D, in some embodiments, the exemplary sensor device 200 receives one or more of pressure stimulus 112D, chemical or biochemical stimulus 114D, and temperature stimulus 116D. In some embodiments, the exemplary sensor device 200 generates an electrical response 118D in response to one or more of the stimuli 112D, 114D and 116D, similarly to operation of the exemplary sensor device 100D.

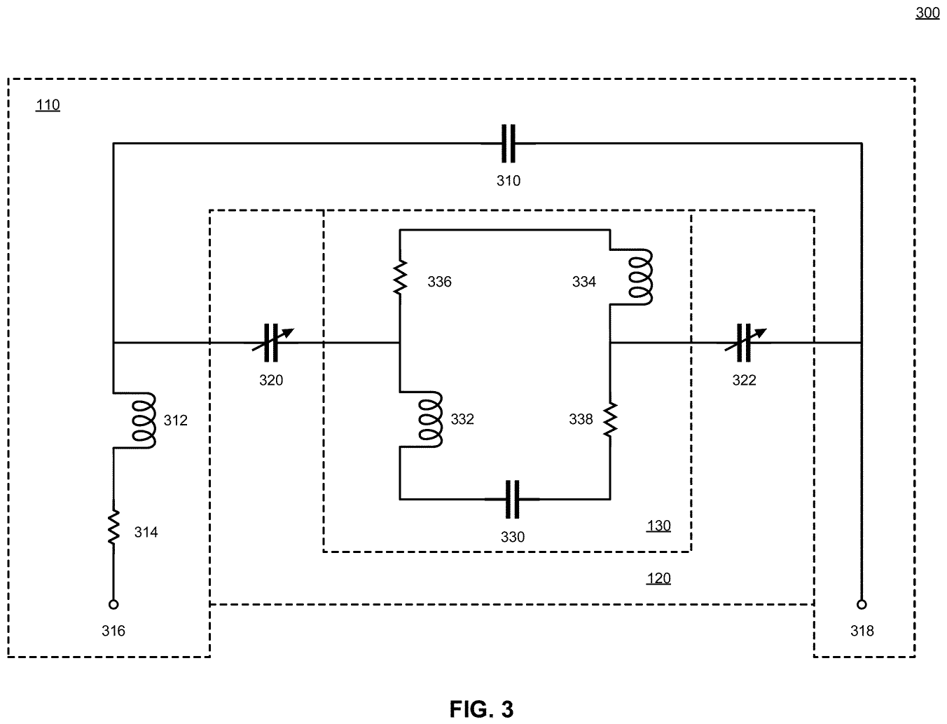

FIG. 3 illustrates an exemplary sensor circuit in accordance with present embodiments. In some embodiments, exemplary circuit 300 includes the upper split-ring metallic structure 110, the lower split-ring metallic structure 130, and the interlayer 120. In some alternative embodiments, the exemplary circuit 300 includes the upper and lower planar-coil metallic structures 210 and 212 and the interlayer 120. In some embodiments, the exemplary circuit 300 includes capacitor 310, inductor 312, resistor 314, and terminals 316 and 318 associated with the upper split-ring metallic structure 110. In some embodiments, the exemplary circuit 300 includes variable capacitors 320 and 330 associated with the interlayer 120. In some embodiments, the exemplary circuit 300 includes inductors 332 and 334, resistors 336 and 338, and capacitor 330 associated with the lower split-ring metallic structure 130.

In some embodiments, an exemplary sensor device 100 or 200 operates as an exemplary series RLC-equivalent circuit including the inductor 312, the resistor 314, and the capacitor 310. In the circuit, capacitance varies with respect to changing physical or dielectric properties of the interlayer 120. Physical or dielectric properties include any response to environmental stimuli, including but not limited to mechanical swelling, structural deformations, and/or biochemical absorption profiles engineered into the interlayer 120. The exemplary sensor device 100 or 200 can be configured to possess particular capacitance and signal specificity by configuring properties of the interlayer 120. In some embodiments, properties of the interlayer 120 include hydrogel stiffness and composition. In some embodiments, the interlayer 120 is configured to be responsive to specific environmental stimuli including, but not limited to, pH, temperature, pressure, salinity, and the like in accordance with present embodiments. High-frequency, mild currents resulting from the exemplary circuit 300 are non-degrading, advantageous for electromagnetic read-out.

The exemplary circuit 300 includes inductors 312, 332 and 334 capacitively coupled via the interlayer 120 and mutual inductance. In its most basic form, it can be modelled as 2 inductors coupled via a single capacitor. In some embodiments, the capacitance of the exemplary circuit 300 is a lossy element possessing a loss tangent depending on the amount of at least one of absorbed water and ions in the interlayer 120. In some embodiments, this loss dominates over resistive losses in the split ring metallic structures 110 and 112. In some embodiments, electrical characteristics of the exemplary sensor device 100 are configured based on one or more of thickness of the interlayer 120, transit of ions into the interlayer 120, replacement of air in a porous matrix of the interlayer 120 with ethanol, oil or the like, and modulation of interlayer permittivity due to the absorption of analyte. These configurable responses can control the range or instantaneous value of variable capacitance of the capacitors 320 and 330, and this an exemplary response of the exemplary sensor device 100 due to various environmental stimuli. In some embodiments, pressure sensing is driven via the compression of the interlayer 120, causing the interlayer 120 to experience both a modulation of interlayer permittivity and a shift in the interlayer thickness. In some embodiments, a reduction in thickness of the interlayer 120 and an increase in antenna size correspond to reduction in the resonant frequency of an exemplary sensor device. Additionally, in some embodiments, exemplary resonant frequencies exhibit a square-root relationship with respect to capacitance of sensor devices in accordance with present embodiments. The terminals 316 and 318 can couple electrically and physically electronic device including one or more of the electronic device 130, the terminal contacts 220 and 222, an antenna capable of RF or other wireless communication, or the like.

In some embodiments, the capacitors 310 and 330 represent capacitive effects at the interface between two respective ends of each of the split-ring metallic structures 110 and 130. In some embodiments, the capacitors 310 and 330 alternatively represent capacitive effects between various parallel lengths of each of the planar-coil metallic structures 210 and 212. In some embodiments, the capacitors 320 and 322 represent independent capacitive effects of separate halves of each of the split-ring metallic structures interacting with a respective portion of the interlayer 120, to form highly sensitive sensors that can be read out remotely via inductive coupling with a remote reader/read-out coil. In accordance with present embodiments, exemplary sensor devices 100 and 200 including RF communication capability are flexibly and stretchably integrable into a multitude of environments, can be read out through opaque mediums, and require no microelectronic components at the sensing node. Further, the sensor read-out is non-degradative as no electrolysis and minimal heat is generated during RF read-out in accordance with present embodiments.

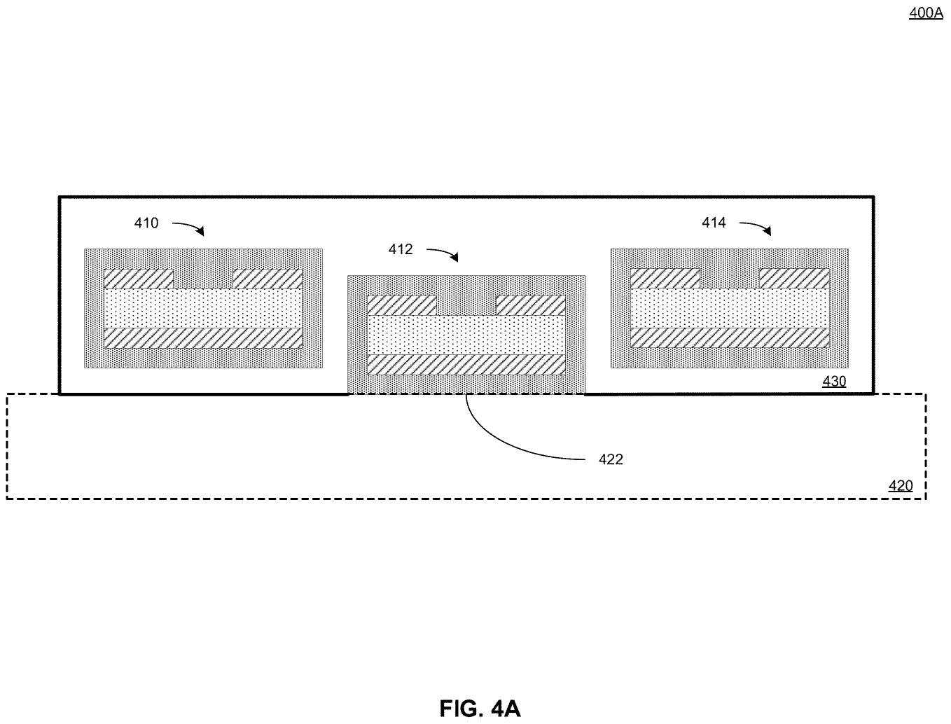

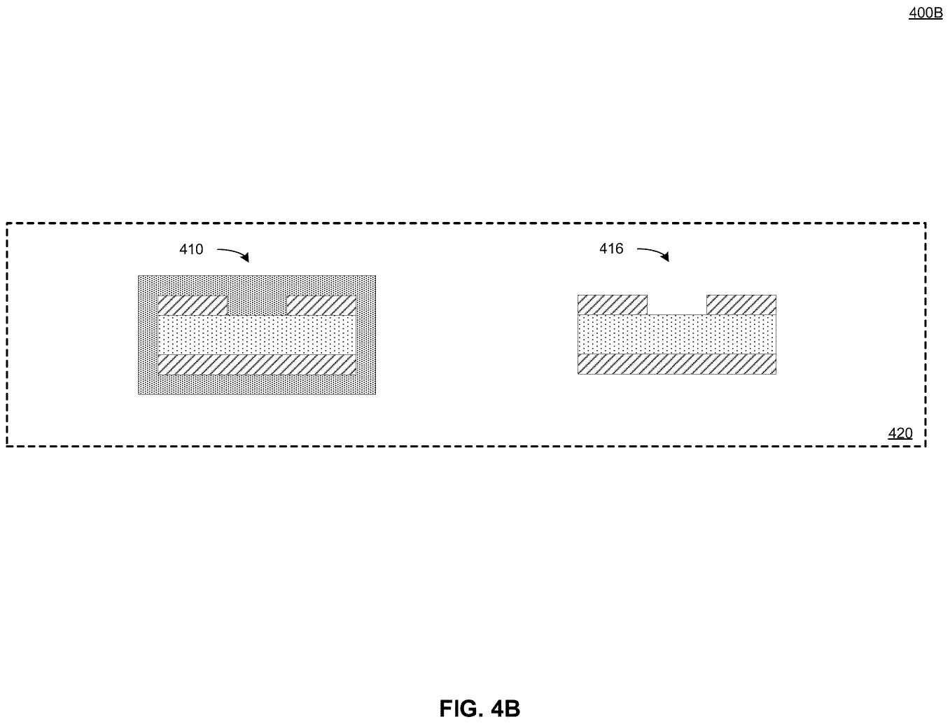

FIG. 4A illustrates an exemplary surface-mountable, wearable, or like sensor system including the exemplary sensor device of FIG. 1A, in accordance with present embodiments. In some embodiments, an exemplary sensor system 400A includes a plurality of sensor devices 410, 412, and 414 in accordance with at least one of the exemplary sensor devices 100 and 200. In some embodiments, the exemplary sensor system 400A includes an encapsulator 430. In some embodiments, the encapsulator 430 includes a cavity 422. In some embodiments, the exemplary sensor system 400A is contactable, in contact, or the like, with a substrate 420. In some embodiments, sensor devices in accordance with sensor devices 100 and 200 are embedded within one or more hydrogel or silicone layers comprising the encapsulator 430. Exemplary embodiments including the encapsulator 430 thus enable stretchable, passive and wireless sensor-skins with extended operational lifetimes.

The sensor devices 410, 412 and 414 include one or more of the sensor devices 100 and 200, and are responsive to one or more environmental stimuli in accordance with present embodiments. These exemplary sensors are embeddable directly into encapsulators 430 that in some embodiments include soft, stretchable formats that blur the line between materials and devices. In some embodiments, the exemplary sensor system 400A is an all-wireless, multi-functional wristband capable of co-monitoring a variety of biometric signals originating along the skin, without any embedded micro-electronics. In some embodiments, an ecoflex or silicone encapsulator 430 completely envelopes sensor devices 410 and 414. In some embodiments, the sensor devices 410 and 414 are respectively responsive to pressure and temperature stimuli, and the encapsulator prevents evaporation of liquid water therein and penetration of other foreign liquids, contaminants, or the like.

In some embodiments, the encapsulator 430 partially envelopes solute-responsive sensor device 412. In some embodiments, the sensor device 412 is disposed within cavity 422 with one face contactably exposed. This way, the exemplary sensor device 412 is both protected from dehydration and contamination by the encapsulator 430, and contactable with substrate 420 to contactably receive at least one of chemical and biochemical stimulus. In some embodiments, the substrate 420 comprises biological skin, and the biochemical and chemical stimuli received by the sensor device 412 include salinity or sweat content of skin. In some embodiments, the substrate 420 comprises a surface of a tooth, and the biochemical and chemical stimuli received by the sensor device 412 include nutrients present in the mouth. In some embodiments, the sensor system 400A passively and wirelessly reports on one or more of pressure, sweat, and temperature on skin without any external power or coupled microelectronics. Sensor devices responsive to pressure, temperature, or the like can be sealed a wireless wristband including hydrogel-interlayer sensors possessing different resonant frequencies and reporting concurrently.

FIG. 4B illustrates an exemplary embeddable, implantable, or like sensor system including the exemplary sensor device of FIG. 1A, in accordance with present embodiments. In some embodiments, an exemplary sensor system 400B includes one or more of sensor devices 410 and 416. In some embodiments, the exemplary sensor system 400B is embeddable, embedded, implantable, implanted, or the like, in the substrate 420. In some embodiments, the sensor device 416 corresponds to the sensor device 100, and lacks an outer hydrogel layer. In some embodiments, the sensor devices 410 and 416 are embedded within a substrate. In some embodiments, the substrate is biological skin, and the sensor system 400B is implanted subdermally within or below the skin. In some embodiments, the sensor system 400B passively and wirelessly reports on one or more of glucose, temperature, or the like, within skin without any external power or coupled microelectronics.

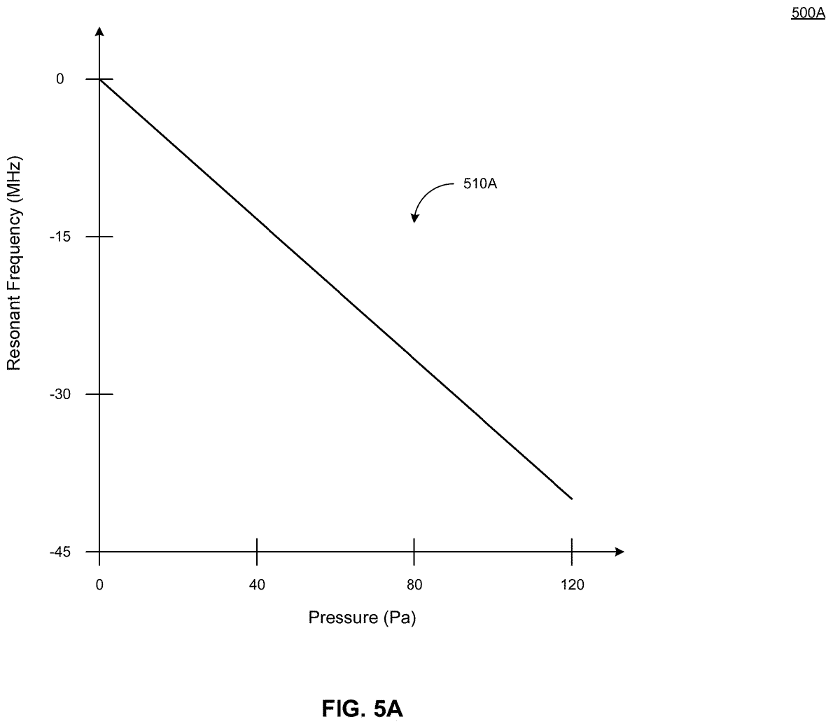

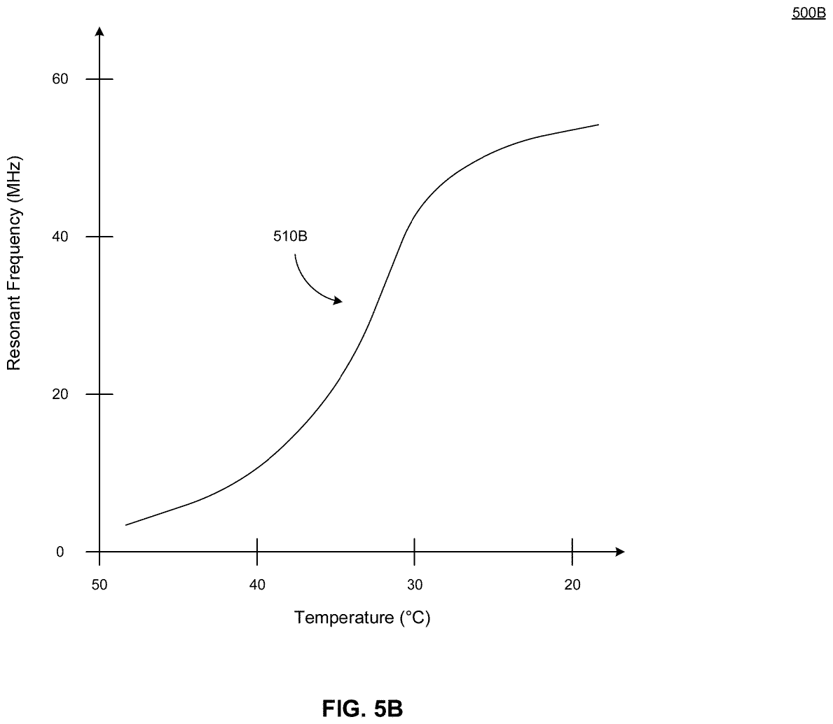

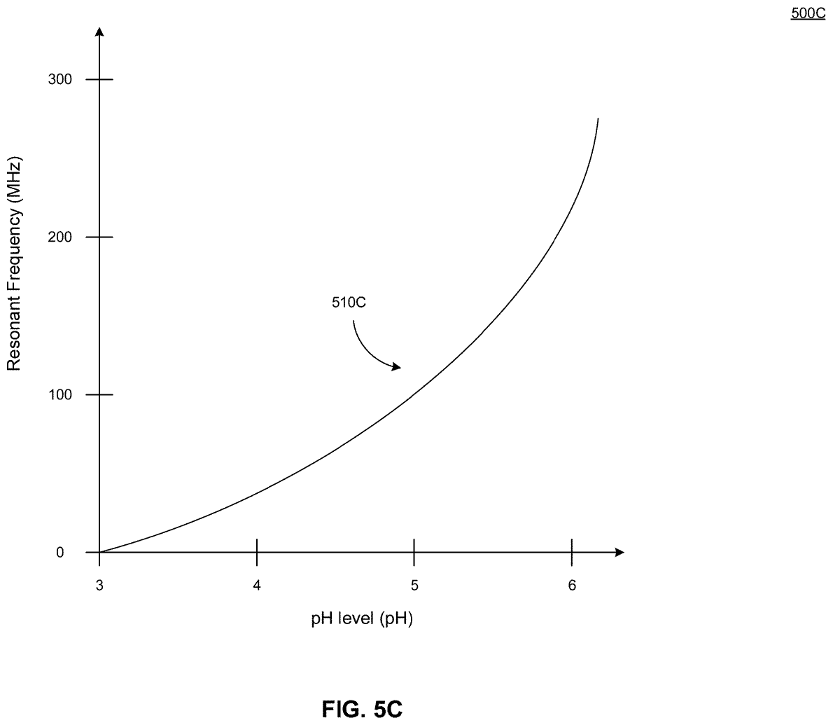

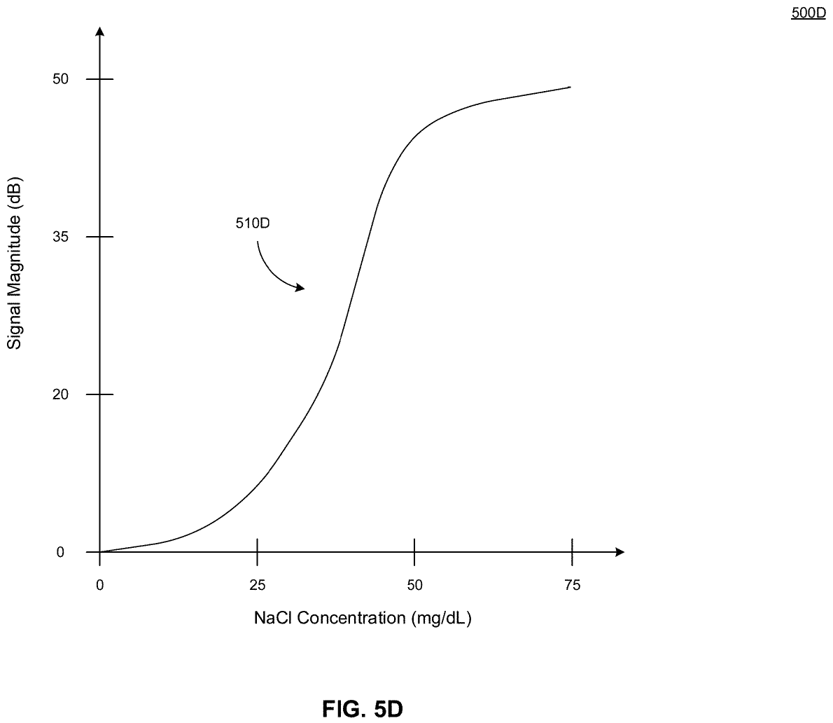

FIG. 5A illustrates an exemplary resonant frequency response to pressure stimulus of an exemplary sensor device in accordance with present embodiments. As illustrated in exemplary chart 500A, in some embodiments, a sensor device in accordance with present embodiments includes an exemplary pressure response 510A. In some embodiments, the exemplary pressure response 510A is a resonant frequency change in response to pressure applied to a sensor device in accordance with present embodiments. In some embodiments, a magnitude of the resonant frequency of an interlayer is inversely proportional to a magnitude of pressure applied to the sensor device including the interlayer.

FIG. 5B illustrates an exemplary resonant frequency response to temperature stimulus of an exemplary sensor device in accordance with present embodiments. As illustrated in exemplary chart 500B, in some embodiments, a sensor device in accordance with present embodiments includes an exemplary temperature response 510B. In some embodiments, the exemplary temperature response 510B is a resonant frequency change in response to temperature change of a sensor device in accordance with present embodiments. In some embodiments, a magnitude of the resonant frequency of an interlayer is nonlinearly proportional to a magnitude of temperature applied to the sensor device including the interlayer.

FIG. 5C illustrates an exemplary resonant frequency response to ionic hydrogen, pH, or like stimulus of an exemplary sensor device in accordance with present embodiments. As illustrated in exemplary chart 500C, in some embodiments, a sensor device in accordance with present embodiments includes an exemplary pH response 510C. In some embodiments, the exemplary pH response 510C is a resonant frequency change in response to change of a concentration of ionic hydrogen in contact with a sensor device in accordance with present embodiments. In some embodiments, a magnitude of the resonant frequency of an interlayer is nonlinearly proportional to a concentration of ionic hydrogen in contact with the sensor device including the interlayer.

FIG. 5D illustrates an exemplary signal magnitude response to salt (NaCl) stimulus of an exemplary sensor device in accordance with present embodiments. As illustrated in exemplary chart 500D, in some embodiments, a sensor device in accordance with present embodiments includes an exemplary salt concentration response 510D. In some embodiments, the exemplary salt concentration response 510D is a signal magnitude change in response to change of a concentration of partially or fully dissolved salt in contact with a sensor device in accordance with present embodiments. In some embodiments, a magnitude of the response signal of an interlayer is nonlinearly proportional to a concentration of salt in contact with the sensor device including the interlayer.

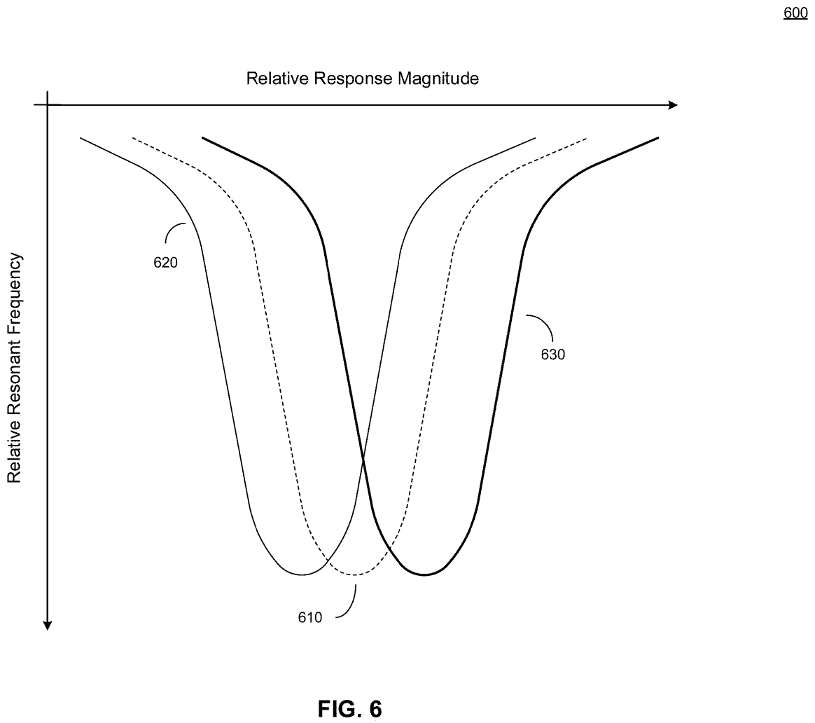

FIG. 6 illustrates an exemplary change to response magnitude relative to resonant frequency, in response to exemplary stimuli of an exemplary sensor device in accordance with present embodiments. As illustrated in exemplary chart 600, in some embodiments, a sensor device in accordance with present embodiments includes at least one of a pre-stimulus response characteristic 610, a pressure stimulus response characteristic 620, and a temperature stimulus response characteristic 630. The pre-stimulus response characteristic 610 represents an expected response range for an exemplary interlayer in accordance with present embodiments, and in the absence of pressure stimulus or temperature stimulus.

The pressure stimulus response characteristic 620 represents a modified expected response of the exemplary interlayer when receiving a pressure stimulus. In some embodiments, hydrogels possess a wide range of elastic moduli from 1 kPa to 500 kPa. In some embodiments, pressure-responsive interlayer materials include relatively soft materials formed from PAM at 2.5% w/w and PEG6000 at 10% w/v, forming exemplary interlayers with elastic moduli in the 20 kPa range. As one example, application of force on an exemplary sensor reduces the distance between respective split-ring metallic structures. The reduction in distance, in turn, increases capacitance and reduces resonant frequency of the sensor device. In some embodiments, shifting thickness of the interlayer modulates sensitivity of the resulting sensor device.

The temperature stimulus response characteristic 630 represents a modified expected response of the exemplary interlayer when receiving a temperature stimulus. In some embodiments, the exemplary interlayer includes basic NIPAM hydrogels, which are only temperature sensitive. Exemplary sensor devices in accordance with present embodiments are responsive at least to temperatures between 25° C. and 45° C., matching optimal thermal range for NIPAM thermo-sensitivity.

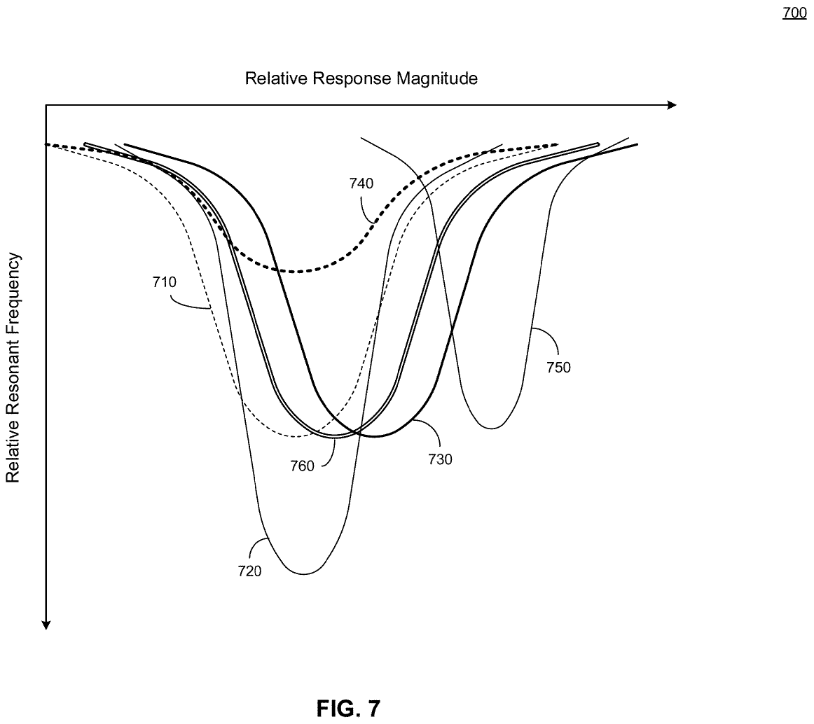

FIG. 7 illustrates an exemplary change to response magnitude relative to resonant frequency, in response to further exemplary stimuli of an exemplary sensor device in accordance with present embodiments. As illustrated in exemplary chart 700, in some embodiments, a sensor device in accordance with present embodiments includes at least one of a pre-stimulus response characteristic 710, a water stimulus response characteristic 720, an oil or ethanol stimulus response characteristic 730, a salt stimulus response characteristic 740, an ionic hydrogen or pH stimulus response characteristic 750, and a glucose stimulus response characteristic 760. In some embodiments, an exemplary sensor device possesses the pre-stimulus response characteristic 710 before introduction of any environmental stimulus or analyte, or in the presence of environmental stimulus or analyte representing a baseline condition or state. Environmental stimuli or analytes in accordance with present embodiments include, but are not limited to, solutions containing mixtures of major nutrients of oils, glucose, salt, or the like. In some embodiments, oils include vegetable oil, ethanol, carbohydrates, and the like. In some embodiments, interlayers of differing composition possess preferential sensitivity to specific nutrients to enable improved classification of various environmental stimuli.

In some embodiments, a sensor device or system in accordance with present embodiments includes an interlayer comprising silk, silk fibroin, or the like. Silk is a hygroscopic biopolymer with both individual properties and properties comparable to biopolymers including cellulose, alginate, hyaluronic acid, gelatin. In some embodiments, due to tight porosity, silk forms a membrane that rejects large molecules, possesses unique absorption characteristics for a variety of molecules, and expands and contracts in thickness in response to certain environmental stimuli. In some embodiments, silk interlayer material is structurally robust and flexible in water and forms compact, mechanically-strong constructs that can be affixed to various substrates to monitor various environmental stimuli. An exemplary sensor device or system in accordance with present embodiments maintains signal permittivity in the presence of water having high permittivity, and thus sensor effectiveness in aqueous environments.

In some embodiments, absorption and swelling characteristics in silk cause a distinctive divergence in resonant frequency response that helps to quantify complex biofluids. In some embodiments, an exemplary sensor device comprising a silk interlayer possesses the water stimulus response characteristic 720 in response to introduction of water comprising environmental stimulus exceeding the baseline condition or state. In this exemplary state, the water stimulus response characteristic possesses a greater magnitude than the pre-stimulus response characteristic 710. In some embodiments, saliva, fatty food/alcoholic drinks, sugary drinks, water, and salty foods exhibit distinct or unique temporal spectral signatures when stimulating an exemplary sensor device in accordance with present embodiments. While many foods exhibit a dominant nutrient characteristic including salt, fat, sugar or the like, an exemplary sensor device in accordance with present embodiments can distinguish simple combinations of foods due to these temporal signatures. As one example, an exemplary in-vivo sensor device affixed to a human tooth detects both salt and fat during exposure to soup during ingestion.

In some embodiments, the water stimulus response characteristic 720 diverges from response characteristics in response to other chemical, biochemical, or like environmental stimuli. In some embodiments, the exemplary sensor device possesses the salt stimulus response characteristic 740 in the presence of salt exceeding the baseline condition or state. In this exemplary state, charged salt molecules penetrate the silk fibroin interlayer and act as shielding for the charged fibroin proteins. This results in a decrease in resonant frequency and reduced amplitude in correlation with salt, due to an increase in charged particles. In some embodiments, the exemplary sensor device possesses the oil or ethanol stimulus response characteristic 730 in the presence of at least one of oil or ethanol exceeding the baseline condition or state. In this exemplary state, molecules of oil, ethanol, alcohol, or the like interact with the hydrophobic portion of silk fibroin to swell the interlayer and replace water therein. This leads to an increase in resonant frequency. In some embodiments, the exemplary sensor device possesses the glucose stimulus response characteristic 760 in the presence of glucose, carbohydrate, or the like exceeding the baseline condition or state. In some embodiments, the exemplary sensor device exhibits a unique temporal response in response to the addition of glucose, carbohydrate, or the like. In this exemplary state, the resonant frequency reduces, before subsequently increasing to its final value above the initial resonant frequency. In some embodiments, this resonant frequency response is due to small molecule osmosis and initial rejection of the molecule by silk.

An implantable and long-term, passive sensor for glucose in accordance with present embodiments is thus advantageously transformative for diabetes treatment. Glucose is a critical metabolite for the human body, and its monitoring has diverse uses from tracking human metabolism to assisting diabetes treatment. In some embodiments, glucose becomes bound into an exemplary interlayer via boronate ester complexes between boronate ions and saccharide diols. In some embodiments, the exemplary interlayer thus undergoes significant swelling in the presence of changing glucose concentrations in blood. In some embodiments, sensitivity of the swelling can be modulated with pH of the hydrogel because sensitivity of swelling due to glucose is heavily dependent on the pH of the environment. In some embodiments, the exemplary interlayer includes a boronic-acid modified hydrogel. In some embodiments, the exemplary interlayer is of mildly swelling varieties based on polyacrylamide-co-polyethyleneglycol diacrylate and alginate. In alternate embodiments, the exemplary interlayer is of more aggressively swelling varieties based on NIPAM. For vinyl-based hydrogel, boronic acid can be co-polymerized via the addition of 3-(Acrylamido)phenylboronic acid into the hydrogel precursor solution. For alginate, boronic acid can be co-polymerized via EDC-NHS coupling between carboxylic acid groups in alginate and 3-aminophenylboronic acid. Boronic acid can be be added to hydrogels at varying weight ratios, and integrated into interlayer-RF architectures.

The ionic hydrogen or pH stimulus response characteristic 750 represents a modified expected response of the exemplary interlayer when receiving an ionic hydrogen stimulus. In some embodiments, ion-selective membranes selectively absorb or transit various charged ions from the environment into its polymeric matrix. Charged ions include, but are not limited to, such as H⁺, Na⁺, Ag⁺, Cl⁻, or the like. In some embodiments, membranes in accordance with present embodiments are integrated onto planar metallic structures of exemplary sensor devices. Exemplary interlayer-RF sensor devices are thus configurably sensitive to a variety of ion sensitivities relevant to human intake, including Sodium, Potassium, Calcium, and Chloride.

In some embodiments, an exemplary interlayer includes a p(NIPAM-co-AA) hydrogel which significantly contracts in the presence of hydrogen ions. Within these exemplary co-polymer gels, the protonation of the carboxylic acid groups modulate the lower critical solution temperature (LCST) of NIPAM, creating dramatic swelling and deswelling with shifts in pH around the pKa of carboxylic acid. In some embodiments, submersion of sensors in a neutral solution (pH 7) shifts the frequency response by approximately 10% from a pH 4 solution. In some embodiments, exemplary sensor devices are most responsive to pH changes between 4 and 6 that correspond, by way of example, to the pKa (˜5) of carboxylic acid. In some embodiments, a thicker interlayer increases ion sensitivity of an exemplary sensor device. In some embodiments, a glucose-responsive hydrogel transitions from swelling to deswelling, and finally swelling again with increasing concentrations of glucose. In alternate embodiments, the exemplary glucose-responsive hydrogel monotonically swells with increasing glucose.

Hydrophobic/oleophilic membranes, in some embodiments, are typically highly porous, possessing greater than 90% air. Various high-permittivity oils displace this air upon contact. In some embodiments, interlayers in accordance with present embodiments include these oil-responsive membranes to monitor oil concentration and measure oil accumulation. The switch in the predominant interlayer material from air to oil reduces the resonant frequency of the exemplary sensor device. In some embodiments, an exemplary sensor device including this exemplary interlayer can directly sense fat intake, monitoring the accumulation of fat in the body, digestion of fats in the intestinal tract, or the like. Sensor in accordance with present embodiments are biocompatible, passive sensors for monitoring environmental stimuli including biochemical state changes within the human body.

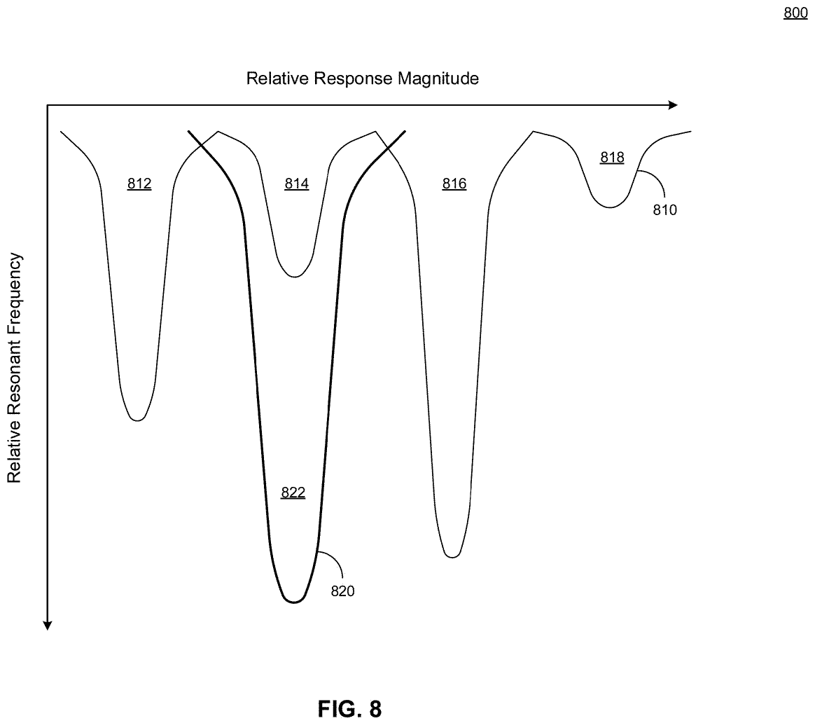

FIG. 8 illustrates an exemplary change to response magnitude relative to resonant frequency, in response to multiple exemplary stimuli of an exemplary sensor system in accordance with present embodiments. In some embodiments, an exemplary sensor system in accordance with present embodiments includes a plurality of sensor devices in accordance with at least of one FIGS. 1A-F, 2A-D and 3. In some embodiments, an exemplary sensor system in accordance with present embodiments includes a sensor system in accordance with at least of one FIGS. 4A-B. As illustrated in exemplary chart 800, in some embodiments, a sensor system in accordance with present embodiments includes at least one of a multi-stimulus response characteristic 810, and a compound-stimulus response characteristic 820.

Sensor devices in accordance with present embodiments are scalable and arrayable. In addition, interlayers of various arrayed sensor devices can be configured to absorb different analytes. In some embodiments, interlayers are configured by utilizing different biopolymers possessing unique side-chains. In alternate embodiments, interlayers are configured by chemically modifying the sidechains of biopolymers. In some embodiments, sensor systems including a plurality of arrayed sensor devices receive complex spectral responses to numerous types and instances of environmental stimuli. In some embodiments, the arrayed responses are analyzed via multi-variate analysis or deep learning to assess complex biofluids such as saliva, blood, or urine. Exemplary sensor arrays are, in some embodiments, passive and wireless, and operate primarily aqueous environments.

Sensor devices in accordance with present embodiments yield unique, powerful functionality when combined and arrayed. In some embodiments, a sensor array includes a compound-stimulus response characteristic 820 amplifying a single environmental stimulus from multiple sensor devices configured to respond to that same stimulus. As one example, an array of a plurality of sensor devices arrayed proximally to each other and configured to respond to a particular glucose concentration, responds with the compounded response peak 822. As another example, an array of a plurality of sensor devices arrayed proximally to each other and configured to respond to a distinct environmental stimuli responds with a multi-stimulus response characteristic 810 including multiple response peaks 812, 814, 816 and 818. In this example, each response peak is configured to a distinct environmental stimulus, and no compounding occurs. In this example, the response peak 814 is an uncompounded response peak for glucose concentration, with a comparatively smaller magnitude that the response peak 822 receiving compounded glucose concentration signal from multiple arrayed sensor devices. It is to be understood that response peaks 812, 814, 816, 818 and 822 can be associated with any environmental stimulus or stimuli in accordance with present embodiments and are not limited to the exemplary stimuli herein.

In some embodiments, arrayed sensor devices in accordance with present embodiments are probed remotely via coils or the like capturing the resonance of multiple sensor devices. In some embodiments, an exemplary sensor array transmits complex one or more multi-stimulus response characteristics to a data processor as is known or may become known. In some embodiments, the data processor includes one or more computational devices capable of multi-variate analysis or deep learning.

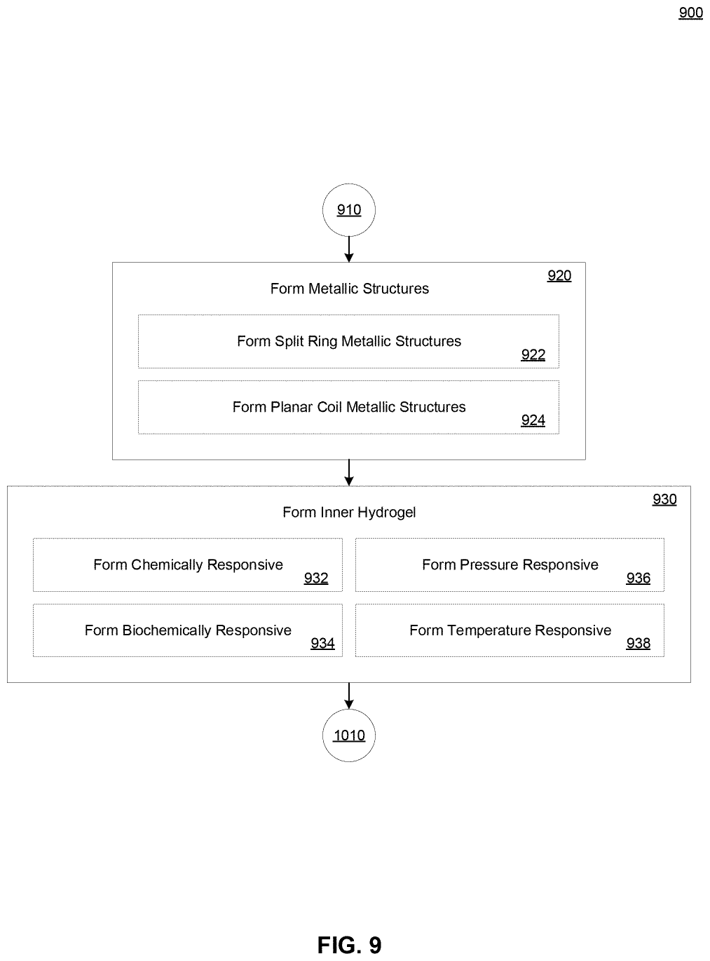

FIG. 9 illustrates an exemplary method of manufacturing an exemplary sensor device in accordance with present embodiments. Method 900 begins at step 910. The method 900 then continues to step 920.

At step 920, one or more metallic structures are formed. In some embodiments, metal conductive sheets are pasted on vinyl, and conductive patterns are created using an electronic cutter. In some embodiments, the conductive sheets include one or more of aluminum and titanium foils. In some embodiments, the step 920 includes at least one of step 922 and step 924. At step 922, split-ring metallic structures are formed. At step 924, planar coil metallic structures are formed. Split-rings or multi-turn planar coils are thus fabricated by vinyl-cutting, microlithography, or like fabrication techniques. In some embodiments, the patterned conductive sheets are temporarily scaffolded to a flexible substrate via degradable films. The conductive material is optionally treated with adhesion promoters (e.g., acrylate-silane). The method 900 then continues to step 930.

At step 930, an inner hydrogel interlayer is formed. In some embodiments, the interlayer 120 includes one or more hydrogel. In some embodiments, hydrogels are highly porous polymers that are primarily water. Exemplary hydrogels possess multiple potential properties, allowing the fabrication of soft-to-stiff, hydrated, porous constructs compatible with biological constructs, properties, or the like. In some embodiments, hydrogels in accordance with present embodiments are engineered to display a wide variety of environmental swelling responses including, but not limited to, sensitivity to pressure, metal ions, pH, temperature, glucose, and more. In some embodiments, hydrogel materials are biocompatible, and is mechanically matched to parameters of living tissues. In some embodiments, the inner hydrogel interlayer possesses a dielectric constant of approximately 80 at radiofrequencies due to its high water content, making the material an ultra-k dielectric or the like. This enables device shrinking while achieving low resonant frequencies required by UHF and NFC RFID reader systems, without coupled micro-electronics.

In some embodiments, an exemplary interlayer is formed from biopolymers with varying absorption characteristics via the integration of different hygroscopic biopolymer films. Hygroscopic biopolymer films include, but are not limited to, silk fibroin, alginate, collagen, cellulose, and the like. In some embodiments, films of material are deposited using spin-coating, and respective split-rings are combined via hydration of films and embossing at elevated temperature. In alternative embodiments, an exemplary interlayer is formed from chemical modification of biopolymers with side-chains modulating the hydrophobicity or hygroscopic nature of the biopolymer. In some embodiments, forming the inner hydrogel interlayer includes binding silk fibroin, to alginate by EDC-NHS conjugation chemistry, or like process. In some embodiments, silk and alginate are bound to hydrophobic side chains or hydrophilic side chains.

In some embodiments, hydrophobic side chains include but are not limited to hexylamine, hexanoic acid, or the like. In some embodiments, hydrophilic side chains include but are not limited to arginine, arginine-rich peptides, or the like. In some embodiments, these chains are introduced in varying amounts to configure chemical or biochemical absorption characteristics, and thus environmental stimulus response characteristics, of an exemplary interlayer in accordance with present embodiments. In some embodiments, interlayers configured with side chains yield interlayers with either preferential sensitivity to charged moieties or hydrophilic compounds, or hydrophobic molecules with a heavy number of carbon chains. By arraying sensors with specifically tuned absorption characteristics, exemplary sensor devices and systems in accordance with present embodiments are configurable to display complex spectral responses in the presence of multiple biofluids. In some embodiments, interlayer materials are integrated into sensor devices by spin-coating polymer on respective surfaces followed by embossing, or drop-casting known volumes of pre-polymer solution to form a specific thickness and allowed to gel. In alternative embodiments, the sensor device form is predefined and liquid pre-polymer is infiltrated fluidically.

In some embodiments, the step 920 includes at least one of step 932, 934, 936 and 938. At step 932, a chemically responsive inner hydrogel interlayer is formed. At step 934, a biochemically responsive inner hydrogel interlayer is formed. In some embodiments, a chemically or biochemically responsive inner hydrogel interlayer includes at least one of a solute-responsive polyethylene glycol (PEG) formed from MW:700 (Modulus ˜500 kPa). In some embodiments, the inner hydrogel interlayer includes pH-responsive co-block-polymers of poly-acrylic acid (NIPAM-PAA). In some embodiments, solute-sensing behavior of exemplary PEG 700 hydrogels does not materially degrade over time, when stored at room temperature. In some embodiments, sensor devices and systems in accordance with present embodiments do not materially degrade when subjected to 60° C. temperatures, an excessive temperature for biological systems, over at least several days. With respect to oil-responsive interlayers, the inner hydrogel interlay is formed, in some embodiments, by cellulose aerogels and modified with either methyl-trichlorosilane or atomic layer deposition of titanium dioxide. In alternate embodiments, the inner hydrogel interlayer is formed by direct synthesis of oleophilic cellulose by modifying cellulose fibers with low surface energy moieties and crosslinking in DMSO. Forming the inner hydrogel interlayer further includes, in some embodiments, a one or more silanization steps to facilitate covalent bonding between membranes and metals.

At step 936, a pressure responsive inner hydrogel is formed. In some embodiments, a pressure responsive inner hydrogel interlayer includes pressure-sensitive polyacrylamide (PAM) with elastic moduli of ˜5 kPa. At step 936, a temperature responsive inner hydrogel is formed. In some embodiments, a temperature responsive inner hydrogel interlayer includes temperature-responsive N-Isopropylacrylamide (NIPAM). In some embodiments, the method 900 then continues to step 1010.

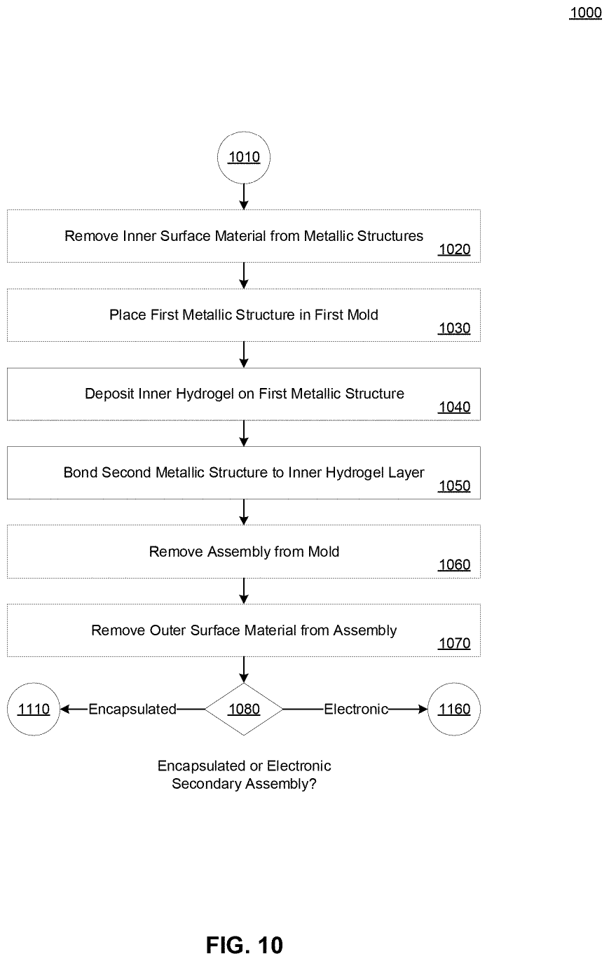

FIG. 10 illustrates an exemplary method of manufacturing an exemplary sensor device further to the exemplary method of FIG. 9. Method 1000 begins at step 1010. The method then continues to step 1020. At step 1020, inner surface materials from one or more of the metallic structures is removed. The method 1000 then continues to step 1030.

At step 1030, the first metallic structure is placed in a first mold. The method 1000 then continues to step 1040.

At step 1040, the inner hydrogel is deposited on a first metallic structure of the metallic structures to form an inner hydrogel layer thereon. In some embodiments, shifting thickness of the interlayer modulates sensitivity of the sensor device. Thus, modulating thickness of sensor devices modulates their optimal performance ranges. The method 1000 then continues to step 1050.

At step 1050, a second metallic structure of the metallic structures is bonded to the inner hydrogel layer. In some embodiments, one or more of ionophore and polymer are dropcast at varying thickness onto one side of the split-ring or planar coil and allowed to dry. Respective ends of the RF-interlayer device are subsequently embossed together to form a single assembly. In some embodiments, the assembly is heat-treated after deposition. In some embodiments, a hydrogel precursor solution is deposited above one half of the resonator and corresponding to the desired hydrogel thickness. In some embodiments, the precursor is deposited before the second half of the resonator is aligned and set above the hydrogel interlayer. The method 1000 then continues to step 1060.

At step 1060, an assembly including the metallic structures and the inner hydrogel layer is removed from the first mold. In some embodiments, after the hydrogel is polymerized at room temperature, the assembly is removed from the first mold. The method 1000 then continues to step 1070.

At step 1070, outer surface material is removed from the assembly. In some embodiments, one or more vinyl backing layers of the metallic structures are removed from the assembly using acetone. In some embodiments, removing backing from and introducing bypass holes into the metallic structures increases mass transport of glucose into the sensor device. The method 1000 then continues to step 1080.

At step 1080, the method 1000 continues to step 1110 to create an encapsulated secondary assembly. Alternatively at step 1080, the method 1000 continues to step 1160 to create an electronic secondary assembly. Further alternatively at step 1080, the method ends if no secondary assembly is to be performed.

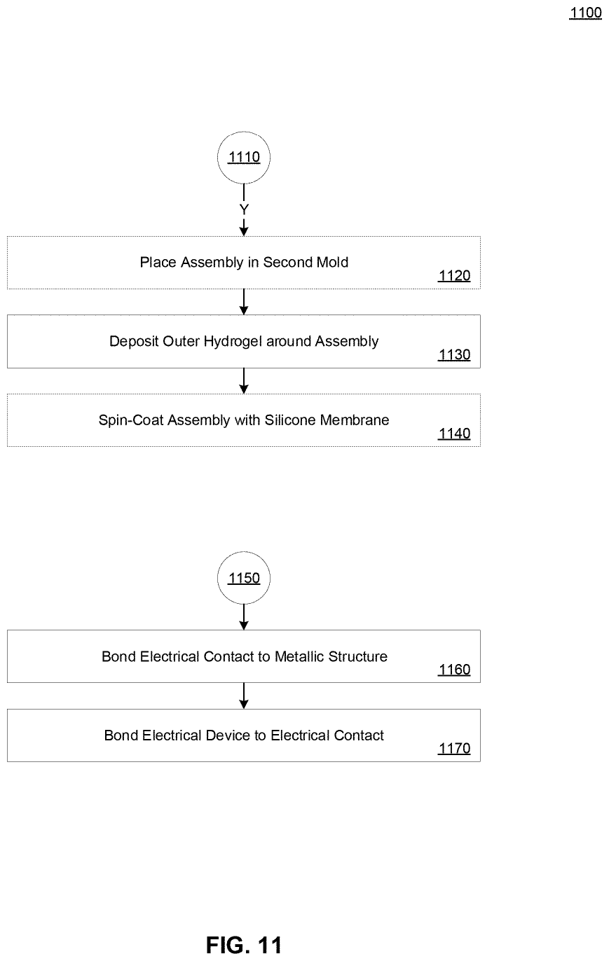

FIG. 11 illustrates an exemplary method of manufacturing an exemplary sensor device further to the exemplary method of FIG. 10. Method 1100 begins at step 1110, for an encapsulated secondary assembly. The method then continues to step 1120. Alternatively, method 1100 begins at step 1150 for an electronic secondary assembly. The method then continues to step 1170.

At step 1120, the assembly is placed in a second mold. The method then continues to step 1130.

At step 1130, an outer hydrogel is deposited around the assembly. In some embodiments, adhesion between an inner hydrogel interlayer and surrounding metallic structures increases by embedding the sensor device assembly directly within one or more further hydrogel layers. Because the metallic layer becomes encompassed by hydrogel in some embodiments, the metal can no longer delaminate from the hydrogel. Thus device failure caused by delamination or other separation of the metallic structures from the interlayer is materially reduced or eliminated. The method then continues to step 1140.

At step 1140, the assembly is coated with a membrane. In some embodiments, the coating process includes spin-coating. In some embodiments, the membrane includes silicone. This encapsulating membrane can be swapped with other selective materials to enhance sensor performance. In some embodiments, silicone membranes protect analytical sensors from dehydration or prevent dehydration altogether by surrounding the material. In some embodiments, silicone membranes are arranged to surround only portions of hydrogel sensors to protect the air-exposed regions. In some embodiments, the method 1100 ends at step 1140.

At step 1160, an electrical contact is bonded to at least one of the metallic structures. In some embodiments, the electrical contact includes an adhesive or the like for affixing an electronic device, antenna, or like structure to the sensor device. The method 1100 then continues to step 1160. At step 1170, an electrical device is bonded to the electrical contact. In some embodiments, the method 1100 ends at step 1140.

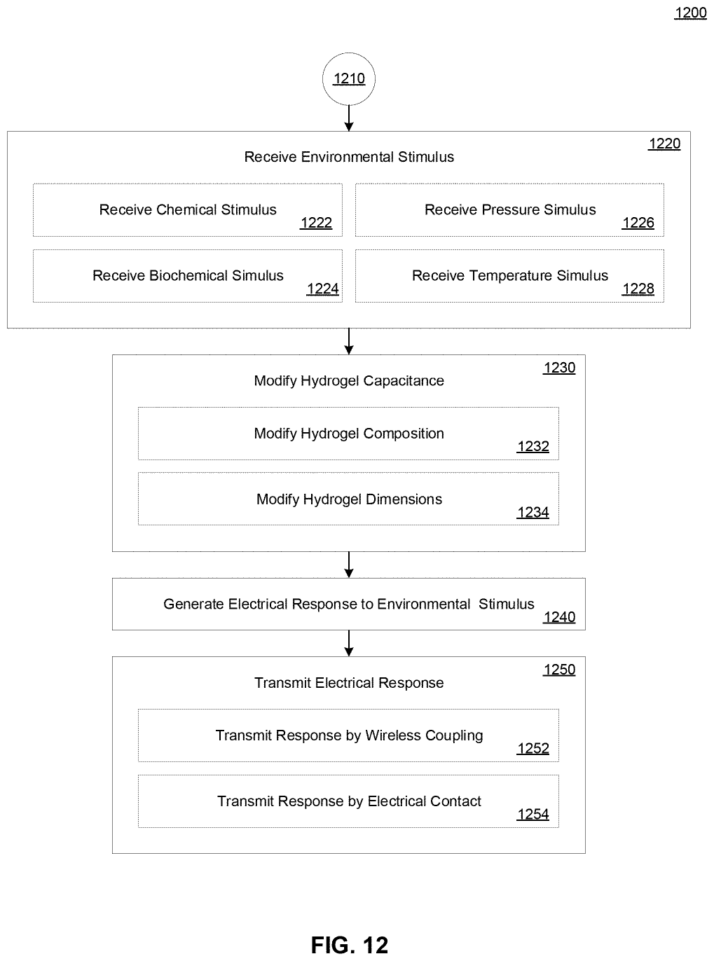

FIG. 12 illustrates an exemplary method of operation of an exemplary sensor device in accordance with present embodiments. An exemplary sensor device, system or array in accordance with present embodiments is compatible with use in a variety of environments. Exemplary compatible environments include but are not limited to in vivo, in vitro, or like biofluids, and food or culture medium monitoring. Compatible biofluids include but are not limited to saliva, urine, sweat, and blood. In some embodiments, RF sensors responsive to chemical or biochemical stimulus operate at around 1 GHz and below. Operation at such exemplary frequencies facilitates sensor read-out by giving access to portable network analyzers and UHF RFID readers. In addition, because water begins to heavily absorb RF signals at 1 GHz and above, sensors operating above these frequencies cannot be implanted too deep in biological tissue. Thus, low operating frequencies, small size, and inexpensive manufacturing are exemplary advantages of sensor device, systems, and arrays in accordance with present embodiments. Method 1200 begins at step 1210. The method then continues to step 1220.

At step 1220, an exemplary sensor device or system in accordance with present embodiments receives at least one environmental stimulus. In some embodiments, the step 1220 includes at least one of step 1222, 1224, 1226 and 1228. At step 1222, the exemplary sensor device or system receives chemical stimulus. At step 1224, the exemplary sensor device or system receives biochemical stimulus. At step 1226, the exemplary sensor device or system receives pressure stimulus. At step 1228, the exemplary sensor device or system receives chemical stimulus. In some embodiments, the exemplary sensor device or system receives a plurality of environmental stimuli comprising a plurality of like or distinct environmental stimuli.

At step 1230, the exemplary sensor device or system modifies capacitance of an inner hydrogel interlayer. In some embodiments, the step 1230 includes at least one of step 1232 and 1234. At step 1232, the exemplary sensor device or system modifies a composition of the inner hydrogel interlayer to modify the capacitance of the inner hydrogel interlayer. At step 1234, the exemplary sensor device or system modifies at least one dimension of the inner hydrogel interlayer to modify the capacitance of the inner hydrogel interlayer. In some embodiments, step 1230 includes both steps 1232 and 1234. As one example, dimensions of the inner hydrogel interlayer may be modified by swelling or contracting in response to biochemical or chemical input. Exemplary biochemical or chemical input causing swelling or contracting includes, but is not limited to, introduction of increased levels of glucose or ionic hydrogen in the presence of the exemplary sensor device or system. As another example, dimensions of the inner hydrogel interlayer may be modified by swelling or contracting based on physical input without chemical or biochemical stimuli by pressure applied to the exemplary sensor device or system. In some embodiments, an exemplary sensor device receives an application of force that reduces the distance between respective faces of a pair of split-ring or planar-coil structures. The reduction in distance, in turn, increases capacitance and reduces resonant frequency of the sensor device.

In some embodiments, interlayers with smaller width, length, or depth swell much faster than larger and thicker counterparts. Thus, further reducing interlayer volume yields quicker and more responsive sensor devices in accordance with present embodiments. The swelling of the hydrogel depends on the pH of the environment, with hydrogel swelling exhibiting an optimal sensitivity at around pH 7 to 7.5. Percent mass of the polymer similarly has an effect on the swelling. In some embodiments, lower percent mass of the polymer exhibits enhanced swelling.

At step 1240, the exemplary sensor device or system generates an electrical response to the environmental stimulus. In some embodiments, phenylboronic acid hydrogels are used for continuous and long-term glucose sensing. In some embodiments, RF read-out generates no destructive electrolysis, and the minimal heat generated during read-out pulses can be readily buffered by the aqueous environment of these sensors.

At step 1250, the exemplary sensor device or system transmits the electrical response. In some embodiments, the step 1250 includes at least one of step 1252 and 1254. At step 1252, the exemplary sensor device or system transmits the electrical response by wireless coupling. At step 1254, the exemplary sensor device or system transmits the electrical response by electrical contact. In some embodiments, the sensor device, system, or array couples to one or more NFC sensor circuits that report on sensor state via a cell phone, wireless computing platform, or the like. In some embodiments, the method 1200 ends at step 1250.

The herein described subject matter sometimes illustrates different components contained within, or connected with, different other components. It is to be understood that such depicted architectures are illustrative, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components and/or wirelessly interactable and/or wirelessly interacting components and/or logically interacting and/or logically interactable components

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general, such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Further, unless otherwise noted, the use of the words “approximate,” “about,” “around,” “substantially,” etc., mean plus or minus ten percent.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

What is claimed is:

1. A sensor device comprising:
an inner hydrogel layer;
a first planar metallic structure adjacent to a first surface of the inner hydrogel layer; and
a second planar metallic structure adjacent to a second surface of the inner hydrogel layer opposite to the first surface.

2. The sensor device of claim 1, wherein the device has a variable capacitance responsive to at least one chemical stimulus applicable to the inner hydrogel layer.

3. The sensor device of any of claims 1 and 2, wherein the device has a variable capacitance responsive to at least one biochemical stimulus applicable to the inner hydrogel layer.

4. The sensor device of any of claims 1-3, wherein the device has a variable capacitance responsive to at least one ethanol stimulus applicable to the inner hydrogel layer.

5. The sensor device of any of claims 1-4, wherein the device has a variable capacitance responsive to at least one glucose stimulus applicable to the inner hydrogel layer.

6. The sensor device of any of claims 1-5, wherein the device has a variable capacitance responsive to at least one ionic hydrogen stimulus applicable to the inner hydrogel layer.

7. The sensor device of any of claims 1-6, wherein the device has a variable capacitance responsive to at least one oil stimulus applicable to the inner hydrogel layer.

8. The sensor device of any of claims 1-7, wherein the device has a variable capacitance responsive to at least one salt stimulus applicable to the inner hydrogel layer.

9. The sensor device of any of claims 1-8, wherein the device has a variable capacitance responsive to at least one water stimulus applicable to the inner hydrogel layer.

10. The sensor device of any of claims 1-9, wherein the sensor device has a variable capacitance responsive to at least pressure stimulus applicable to the inner hydrogel layer.

11. The sensor device of any of claims 1-10, wherein the sensor device has a variable capacitance responsive to at least touch stimulus applicable to the inner hydrogel layer.

12. The sensor device of any of claims 1-11, wherein the sensor device has a variable capacitance responsive to at least temperature stimulus applicable to the inner hydrogel layer.

13. The sensor device of any of claims 1-12, further comprising:
an encasing layer at least partially enclosing at least one of the inner hydrogel layer, the first planar metallic structure, and the second planar metallic structure.

14. The sensor device of claim 13, wherein the encasing layer includes a cavity at least partially exposing a sensing surface of the first planar metallic structure or the second planar metallic structure.

15. The sensor device of any of claims 13 and 14, further comprising:
a membrane at least partially enclosing the encasing layer.

16. The sensor device of any of claims 13-15, wherein the encasing layer comprises a hydrogel.

17. The sensor device of any of claims 13-16, wherein the encasing layer comprises a biopolymer.

18. The sensor device of any of claims 13-17, wherein the encasing layer comprises an elastomer.

19. The sensor device of any of claims 13-18, wherein the encasing layer comprises silicone.

20. The sensor device of any of claims 13-19, wherein the encasing layer includes a cavity at least partially exposing a sensing surface of the first planar metallic structure or the second planar metallic structure.

21. The sensor device of claim 20, wherein the sensing surface is operatively contactable with a surface of biological skin.

22. The sensor device of any of claims 1-21, wherein the device is implantable in living tissue.

23. The sensor device of any of claims 1-22, wherein the device is implantable subdermally.

24. The sensor device of any of claims 1-19, further comprising:
an electronic device operatively coupled to the first metallic structure.

25. The sensor device of claim 24, wherein the first metallic structure comprises a plurality of terminals coupled respectively to terminals of the electronic device.

26. The sensor device of any of claims 1-25, wherein the first planar metallic structure and second planar metallic structure respectively comprise a first split ring structure and a second split ring structure.

27. The sensor device of any of claims 1-26, wherein the first planar metallic structure and second planar metallic structure respectively comprise a first coil ring structure and a second coil ring structure.

28. A method comprising:
receiving at least one environmental stimulus;
modifying a capacitance of a hydrogel in response to the received environmental stimulus; and
generating an electrical stimulus response based on the modified capacitance.

29. The method of claim 28, wherein the environmental stimulus comprises chemical stimulus.

30. The method of any of claims 28 and 29, wherein the environmental stimulus comprises biochemical stimulus.

31. The method of any of claims 28-30, wherein the environmental stimulus comprises pressure stimulus.

32. The method of any of claims 28-31, wherein the environmental stimulus comprises temperature stimulus.

33. The method of any of claims 28-32, wherein the modifying the capacitance of the hydrogel comprises modifying a composition of the hydrogel.

34. The method of claim 33, wherein the modifying the composition of the hydrogel comprises absorbing a chemical.

35. The method of any of claims 33 and 34, wherein the modifying the composition of the hydrogel comprises absorbing a biochemical.

36. The method of any of claims 28-35, wherein the modifying the capacitance of the hydrogel comprises modifying at least one dimension of the hydrogel.

37. The method of any of claims 28-36, wherein the modifying the at least one dimension of the hydrogel comprises expanding the hydrogel.

38. The method of any of claims 28-37, wherein the modifying the at least one dimension of the hydrogel comprises compressing the hydrogel.

39. The method of any of claims 28-38, further comprising:
generating an electrical response to the environmental stimulus.

40. The method of any of claims 28-39, further comprising:
transmitting the electrical response by one or more of wireless coupling and electrical contact.

41. The method of any of claims 28-40, wherein the modifying the capacitance of the hydrogel comprises modifying a resonant frequency of the hydrogel.

42. The method of claim 41, wherein the environmental stimulus comprises a pressure stimulus, and the modifying the resonant frequency of the hydrogel comprises modifying the resonant frequency based on a magnitude of the pressure stimulus.

43. The method of any of claims 41 and 42, wherein the environmental stimulus comprises a temperature stimulus, and the modifying the resonant frequency of the hydrogel comprises modifying the resonant frequency based on a magnitude of the temperature stimulus.

44. The method of any of claims 28-43, wherein the modifying the capacitance of the hydrogel comprises modifying a signal magnitude response of the hydrogel.

45. The method of claim 44, wherein the environmental stimulus comprises a chemical stimulus, and the modifying the signal magnitude response of the hydrogel comprises modifying the signal magnitude based on the chemical stimulus.

46. The method of any of claims 44 and 45, wherein the environmental stimulus comprises a biochemical stimulus, and the modifying the signal magnitude response of the hydrogel comprises modifying the signal magnitude based on the biochemical stimulus.

47. The method of any of claims 44-46, wherein the environmental stimulus comprises an ionic hydrogen stimulus, and the modifying the signal magnitude response of the hydrogel comprises modifying the signal magnitude based on a concentration of the ionic hydrogen stimulus.

48. The method of any of claims 44-47, wherein the environmental stimulus comprises a salt stimulus, and the modifying the signal magnitude response of the hydrogel comprises modifying the signal magnitude based on a concentration of the salt stimulus.

49. The method of any of claims 44-48, wherein the modifying the signal magnitude response of the hydrogel comprises shifting the signal magnitude response relative to the resonant frequency of the hydrogel.

50. The method of any of claims 44-49, wherein the modifying the signal magnitude response of the hydrogel comprises increasing the signal magnitude response relative to the resonant frequency of the hydrogel.

51. The method of any of claims 44-50, wherein the modifying the signal magnitude response of the hydrogel comprises decreasing the signal magnitude response relative to the resonant frequency of the hydrogel.

52. The method of any of claims 44-51, wherein the modifying the signal magnitude response of the hydrogel comprises increasing the signal magnitude response relative to the resonant frequency of the hydrogel, in response to increasing temperature stimulus.

53. The method of any of claims 44-52, wherein the modifying the signal magnitude response of the hydrogel comprises decreasing the signal magnitude response relative to the resonant frequency of the hydrogel, in response to increasing pressure stimulus.

54. The method of any of claims 44-53, wherein the modifying the signal magnitude response of the hydrogel comprises increasing the signal magnitude response.

55. The method of any of claims 44-54, wherein the modifying the signal magnitude response of the hydrogel comprises decreasing the signal magnitude response.

56. The method of any of claims 44-55, wherein the modifying the signal magnitude response of the hydrogel comprises increasing the signal magnitude response, in response to increasing water stimulus.

57. The method of any of claims 44-56, wherein the modifying the signal magnitude response of the hydrogel comprises decreasing the signal magnitude response, in response to increasing salt stimulus.

58. The method of any of claims 44-57, wherein the modifying the signal magnitude response of the hydrogel comprises increasing the signal magnitude response relative to the resonant frequency of the hydrogel by a first shift magnitude, in response to increasing glucose stimulus.

59. The method of any of claims 44-58, wherein the modifying the signal magnitude response of the hydrogel comprises increasing the signal magnitude response relative to the resonant frequency of the hydrogel by a second shift magnitude, in response to increasing oil stimulus.

60. The method of any of claims 44-59, wherein the modifying the signal magnitude response of the hydrogel comprises increasing the signal magnitude response relative to the resonant frequency of the hydrogel by a second shift magnitude, in response to increasing ethanol stimulus.

61. The method of any of claims 44-60, wherein the modifying the signal magnitude response of the hydrogel comprises increasing the signal magnitude response relative to the resonant frequency of the hydrogel by a third shift magnitude, in response to increasing ionic hydrogen stimulus.

62. The method of any of claims 28-61, wherein the receiving the at least one environmental stimulus comprises receiving a plurality of environmental stimuli.

63. The method of claim 62, wherein the receiving the plurality of environmental stimuli comprises receiving a first environmental stimulus, and a second environmental stimulus at least partially distinct from the first environmental stimulus.

64. The method of claim 62, wherein the receiving the plurality of environmental stimuli comprises receiving a first environmental stimulus and a second environmental stimulus at least partially corresponding to the first environmental stimulus.

65. The method of any of claims 62-64, wherein the modifying the capacitance of the hydrogel comprises modifying the capacitance of the hydrogel in response to the first environmental stimulus and the second environmental stimulus.

66. The method of any of claims 62-65, wherein the hydrogel comprises a plurality of hydrogels.

67. The method of any of claims 62-66, wherein the receiving the first environmental stimulus comprises receiving the first environmental stimulus at a first hydrogel.

68. The method of any of claims 62-67, wherein the receiving the second environmental stimulus comprises receiving the second environmental stimulus at a second hydrogel.

69. A method comprising:
forming a plurality of planar metallic structures;
forming an inner hydrogel; and
affixing the inner hydrogel between the planar metallic structures.

70. The method of claim 69, wherein the metallic structures comprise split ring metallic structures.

71. The method of any of claims 69 and 70, wherein the metallic structures comprise planar coil metallic structures.

72. The method of any of claims 69-71, wherein the forming the inner hydrogel comprises forming the inner hydrogel responsive to chemical stimulus.

73. The method of any of claims 69-72, wherein the forming the inner hydrogel comprises forming the inner hydrogel responsive to biochemical stimulus.

74. The method of any of claims 69-73, wherein the forming the inner hydrogel comprises forming the inner hydrogel responsive to pressure stimulus.

75. The method of any of claims 69-74, wherein the forming the inner hydrogel comprises forming the inner hydrogel responsive to temperature stimulus.

76. The method of any of claims 69-75, further comprising:
removing a material from a first surface of at least one of the planar metallic structures.

77. The method of any of claims 69-76, wherein the plurality of planar metallic structures comprise a first planar metallic structure and a second planar metallic structure.

78. The method of claim 77, further comprising:
placing the first planar metallic structure in a first mold.

79. The method of any of claims 77 and 78, wherein the forming the inner hydrogel further comprises depositing the inner hydrogel on the first planar metallic structure.

80. The method of any of claims 77-79, further comprising:
bonding the second planar metallic structure to the inner hydrogel.

81. The method of any of claims 77-80, further comprising:
removing the planar metallic structures and the inner hydrogel from the first mold to obtain an assembly.

82. The method of claim 81, further comprising:
removing outer surface material from the assembly.

83. The method of any of claims 81 and 82, further comprising:
placing the assembly in a second mold.

84. The method of any of claims 69-83, further comprising:
forming an outer hydrogel at least partially surrounding at least one of the plurality of planar metallic structures and the inner hydrogel.

85. The method of claim 84, wherein the forming the outer hydrogel further comprises depositing the outer hydrogel at least partially surrounding at least one of the plurality of planar metallic structures and the inner hydrogel.

86. The method of any of claims 69-85, further comprising:
forming membrane at least partially surrounding at least one of the plurality of planar metallic structures and the inner hydrogel.

87. The method of claim 86, wherein the forming the membrane further comprises at least partially spin-coating, with the membrane, at least one of the plurality of planar metallic structures and the inner hydrogel.

88. The method of any of claims 69-87, further comprising:
embedding, at least partially, the first planar metallic structure, the second planar metallic structure, and the inner hydrogel in a substrate.

89. The method of any of claims 69-88, further comprising:
bonding an electrical device to the first planar metallic structure.

90. The method of claim 89, wherein the bonding the electrical device comprises bonding the electrical device to the first planar metallic structure via an electrical contact.
